(19) United States
(12) Patent Application Publication
(10) Pub. No.: US 2014/0017797 A1
(43) Pub. Date: Jan. 16, 2014

(54) PROTEIN AFFINITY TAG AND USES THEREOF

(71) Applicant: Jamila Willhelmina Zomer-Elhadari, Zeist (NL)

(72) Inventors: Geert Petrus Maria Mommen, Sevenum (NL); Martinus Richardus Jozef Hamzink, De Bilt (NL); Gijsbert Zomer, Zeist (NL); Adrianus Petrus Josephus Maria De Jong, Nieuwkoop (NL); Hugo Derk Meiring, Leusden (NL)

(73) Assignee: De Staadt der Nederlanden, vert doorde minister van VWS, Den Haag (NL)

(21) Appl. No.: 13/993,644

(22) PCT Filed: Dec. 13, 2011
(86) PCT No.: PCT/NL2011/050848

§ 371 Date: Aug. 23, 2013

Related U.S. Application Data
(60) Provisional application No. 61/422,378, filed on Dec. 13, 2010.

(30) Foreign Application Priority Data
Dec. 13, 2010 (EP) 10194794.3
Mar. 11, 2011 (EP) 11157903.3

(51) Int. Cl. C07F 9/572; C07K 1/107; C07F 9/09

(52) U.S. Cl. CPC C07F 9/5723; C07F 9/09; C07K 1/1077
USPC 436/86; 558/170; 530/344; 530/345; 558/175; 558/178; 548/413; 530/300; 428/34.1

(57) ABSTRACT

This invention concerns isotopically coded or non-isotopically coded affinity-tags for analysis of certain target molecules in complex samples, in particular for mass spectrometric analysis of proteomic samples. The affinity-tags have the following general formula X-SPACER-OPO₃H₂, wherein X is a functional group or moiety capable of reacting with a functional group of a protein, peptide, DNA, lipid, sugar and/or steroid. These phosphate affinity tags (‘PTAG’) are capable of high but reversible binding to metal-oxides like TiO₂. Due to this property, tagged sample fractions can be isolated from non-tagged sample fraction by affinity chromatography. The binding of organophosphate to metal-oxides remains intact during multiple washings of preferably acidic solutions to remove non-specifically bound components. PTAG's are also envisaged wherein X is selected such that it is capable of binding proteins, peptides, nucleic acid molecules, lipids, carbohydrates, steroids and the like.

### FIELD OF THE INVENTION

This invention concerns isotopically coded or non-isotopically coded affinity-tags for analysis of certain target molecules in complex samples, in particular for mass spectrometric analysis of proteomic samples. This invention provides novel affinity tags, as well as their preparation and use.

### BACKGROUND OF THE INVENTION

A key aspect to comprehensive characterization of proteomic samples is the quantitative analysis of protein profiles. For this, two alternative approaches are common nowadays. The first method is based on high resolution two-dimensional electrophoresis (2DE) and mass spectrometry, the second on quantitative mass spectrometry via stable isotope tagging of proteins.

In the latter method samples are usually enzymatically digested into peptides by a protease, such as trypsin, and analyzed and identified by Mass Spectrometry (MS). Resulting peptide mixtures are in general highly complex because each protein can generate dozen of peptides. Due to this complexity, information about the substoichiometric peptides is missed because the detection of low abundant peptides is seriously suppressed by the high abundant peptides. To increase in-depth protein identification, most proteome approaches aim to reduce sample complexity. Sufficient reduction (two orders of magnitude) of sample complexity can be achieved when only a representative subset of peptides is selected for analysis. This is accomplished using ‘affinity enrichment’, which involves the chemical coupling of affinity tags to subclasses of peptides which are then enriched using e.g. solid phase resins. Peptides carrying rare amino acids, like cysteins (Cys), methioine (Met), tryptophan (Trp) and histidine (His) are the likely targets for affinity enrichment because a digest of a given protein contains only a few of these specific peptides.

Affinity-Tags are capable of high but specific binding to reactive functional groups of amino acid side chains. Due to this property, specific subsets of peptides can be isolated from the protein digest using special types of affinity tags which differ in residue reactive functional group.

In literature, numerous strategies have been developed to specifically enrich subsets of peptides using affinity tags, including Isotope Coded Affinity Tag (Gygi et. Al. 1999), Fluorous affinity tags (Brittain et. Al., 2005) and Combined Fractional Diagonal Chromatography (COFRADIC) (Gevaert et. Al. 2003).

Besides specific isolation of subsets op peptides, most affinity tags comprise one or more isotope tags allowing for quantitative analysis of complex proteomic samples by using stable isotope variants (¹³C, ²H incorporations). In practice, two or more proteins samples which need to be compared are reacted with chemically identical but isotopically different affinity tags. After that, samples are combined, proteolytically treated and enriched using affinity chromatography. After the bound fragments are eluted from the solid phase resin, the eluates are analyzed by Liquid Chromatography coupled to Mass Spectrometry (LC-MS). Because the affinity tags are chemically identical, the isotopic pair of peptides elute nearly simultaneously from the LC system, however, they differ in mass due to the unique stable isotope pattern of the affinity labels. Information about relative protein concentrations in the two samples can be obtained directly by comparison of the peak areas.

As will be recognized by those skilled in the art, the application of affinty tags is not restricted to protein analysis. Affinity tags are also commonly employed for analysing DNA, lipids, carbohydrates, steroids in complex samples, relying largely on the same principles.

Affinity tags ideally should have a number of physical and chemical properties. First of all, an affinity tag should be capable of high but reversible binding with a solid phase resin to allow for separation of tagged and non-tagged substances. An affinity tag preferably is also sufficiently water-soluble, such that chemical coupling reactions can be carried out in aqueous environments and the affinity tag will not or hardly bind to surfaces of reaction vessels or other sample preparation materials. An affinity tag preferably has low reactivity and, ideally, is relatively small. Furthermore the presence of a suitable type and number of atoms for incorporation of heavy isotope(s) sufficient for MS analysis is highly desirable. Last but not least an affinity tag should be compatible with LC-MS in terms of chromatographic separation and mass spectrometric detection.

It is an objective of the present invention to provide novel and improved affinity tags, especially affinity tags that, by virtue of one or more of the aforementioned properties, facilitate an improvement in the analysis of protein containing samples, especially in the quantitative analysis of proteomic samples.

### SUMMARY OF THE INVENTION

The present inventors surprisingly found that this objective is realized by affinity-tags comprising an organophosphate, said affinity tag having the following general formula:

X—SPACER-OPO₃H₂

wherein X is a functional group or moiety capable of reacting with a functional group of a protein, peptide, DNA, lipid, sugar and/or steroid.

The phosphate affinity tags (‘PTAG’) of the invention are capable of high but reversible binding to metal-oxides like TiO₂. Due to this property, tagged sample fractions can be isolated from non-tagged sample fraction by affinity chromatography. The binding of organophosphate to metal-oxides remains intact during multiple washings of preferably acidic solutions to remove non-specifically bound components.

The phosphate affinity tags are highly soluble. Due to this property, chemical coupling reactions can be performed in aqueous environments ideally for protein en peptide analysis. Organic modifiers are not needed and possible side reactions are largely prevented. Finally, due to the high solubility, the PTAG bind not or minimally to surfaces of reaction vessels or other sample preparation materials.

Furthermore, the methodology using PTAG's is highly compatible with reversed phase LC-MS were samples are retained using aqueous buffers and subsequently eluted using organic buffers, generally acetonitrille.

The organo-phosphate functional group is inert, meaning that the affinity tag does not participate/interfere with any of chemical reactions used to couple the affinity tag to the peptide/protein sample.

The present PTAG's are relative small molecules and could therefore possibly more easily target residues that are sterically hindered compared to bulky affinity tags.

Although present PTAG's undergo hydrophobic interaction with reversed phase chromatography materials, increases in retention shift are relatively minor. This means that PTAG modified sample fractions are found in the same elution window of unmodified sample fractions, and are therefore highly compatible with conventional reversed phase LC systems. Moreover, due to this high compatibility, there is no need to remove the organophosphate group to achieve efficient reversed phase LC separations or optimize the LC separation conditions.

Compatibility with LC-MS in combination with the possibility to integrate metal-oxide like TiO2 into conventional LC-MS systems, opens the possibility to isolation of PTAG modified sample fractions from untagged sample fractions in an automated 2-dimensional LC-MS platform. These automated analytical platforms are of advantage in terms of sample preparation time and possible sample losses.

The presence of isotope tags in the invented PTAG's allows for quantitative analysis of two or more samples according the unique stable isotope pattern in the mass spectrum. Due to this property, specific subsets of protein related samples can be isolated using special types of affinity tags which differ in residue reactive functional group.

As will be recognized by those skilled in the art, the application of the present PTAG's is not restricted to protein analysis. PTAG's are also envisaged wherein X is selected such that it is capable of binding nucleic acid molecules, lipids, carbohydrates, steroids and the like.

Here, the novel affinity tags, as well as their preparation and use is described.

### DETAILED DESCRIPTION OF THE INVENTION

Hence, a first aspect of the invention concerns organophosphate compounds, also referred to herein as ‘phospho-affinity tag’ or ‘PTAG’, selected from the group of compounds having the general structure:

X—SPACER-O—PO₃H₂

wherein:
X is a reactive group which is reactive towards a chemical moiety on a protein, a peptide, a nucleic acid molecule, a lipid, a carbohydrate and/or a steroid, preferably X is a thiol reactive group, an amino reactive group, a carboxylic acid reactive group, a hydroxyl reactive group, or an aldehyde or ketone reactive group; and ‘Spacer’ represents a chain consisting of 1-5 building blocks selected from:
—(CH₂)ₙ—, wherein n is an integer of 1-5 and wherein one or more hydrogen atoms may have been replaced with a substituent independently selected from, —R, —OR, ═O, ═NR, ═N₂, ═N—O—R, —NRR′, —SR, -halogen, —OC(O)R, —C(O)R, —CO₂R, —CONRR′, —OC(O)NRR′, —NRC(O)R′, —NR—C(O)NR″, R′″, —NC(O)₂R, —NR—C(NRR″R′″)═NR″″, —NR—C(NRR″)═NR′″, —SOR, —S(O)₂R, —S(O)₂NRR″, —NRSO₂R′, —CN, —O—PO₃H₂ and —NO₂, wherein R, R′, R″ and R′″ independently represent hydrogen or a lower branched or linear alkyl, alkenyl, alkynyl, alkoxy, thioalkoxy, heteroalkyl, cycloalkyl, cycloalkenyl, aryl, cycloalkylalkyl, cycloalkenylalkyl, or arylalkyl moiety; or with a fluorophore or a chromophore;
-Phe-, wherein one or more hydrogen atoms may have been replaced with a substituent selected from —R, —OR, —NRR′, —SR, -halogen, —OC(O)R, —C(O)R, —CO₂R, —CONRR′, —OC(O)NRR′, —NRC(O)R′, —NR—C(O)NR″, R′″, —NC(O)₂R, —NR—C(NRR″R′″)═NR″″, —NR—C(NRR″)═NR′″, —SOR, —S(O)₂R, —S(O)₂NRR″, —NRSO₂R′, —CN, —O—PO₃H₂ and —NO₂, wherein R, R′, R″ and R′″ independently represent hydrogen or a lower branched or linear alkyl, alkenyl, alkynyl, alkoxy, thioalkoxy, heteroalkyl, cycloalkyl, cycloalkenyl, aryl, cycloalkylalkyl, cycloalkenylalkyl, or arylalkyl moiety;
—CH═CH—, wherein one or two hydrogen atoms may have been replaced with a substituent selected from —R, —OR, —NRR′, —SR, -halogen, —OC(O)R, —C(O)R, —CO₂R, —CONRR′, —OC(O)NRR′, —NRC(O)R′, —NR—C(O)NR″, R′″, —NC(O)₂R, —NR—C(NRR″R′″)═NR″, —NR—C(NRR″)═NR′″, —SOR, —S(O)₂R, —S(O)₂NRR″, —NRSO₂R′, —CN, —O—PO₃H₂ and —NO₂, wherein R, R′, R″ and R′″ independently represent hydrogen or a lower branched or linear alkyl, alkenyl, alkynyl, alkoxy, thioalkoxy, heteroalkyl, cycloalkyl, cycloalkenyl, aryl, cycloalkylalkyl, cycloalkenylalkyl, or arylalkyl moiety;
—C≡C—;
—O—;
—S—; and
—NR—, wherein R indepenently represents hydrogen or a lower branched or linear alkyl, alkenyl, alkynyl, alkoxy, thioalkoxy, heteroalkyl, cycloalkyl, cycloalkenyl, aryl, cycloalkylalkyl, cycloalkenylalkyl, or arylalkyl moiety;
   and, optionally, a building block selected from:
—C(O)—O—SiRR′— and —O—SiRR′, wherein R, and R′ independently represent hydrogen or a lower branched or linear alkyl, alkenyl, alkynyl, alkoxy, thioalkoxy, heteroalkyl, cycloalkyl, cycloalkenyl, aryl, cycloalkylalkyl, cycloalkenylalkyl, or arylalkyl moiety;
—S—S—
—O—PO₂—O—
—S(═O)(═O)—
   or ‘Spacer’ represents a moiety comprising:
one or multiple nucleic acid bases;
a protease or enetrokinase cleavage sequence;
a lactamase sensitive P-lactam analogue;
a thrombin cleavage sequence;
a glycosidase-cleavable sugar; or
a nuclease cleaving sequence;
   wherein the spacer may comprise an optically detectable moiety as a substituent; and salts and esters of said organophosphate compound.

In a particularly preferred embodiment of the invention, a PTAG as defined in any of the foregoing are provided, wherein at least one atom in at least one of the phosphate, X and Spacer moieties has the form of a stable isotope of the corresponding element; and salts and esters thereof.

In a particularly preferred embodiment of the invention, a PTAG as defined in any of the foregoing are provided, wherein at least one of the phosphate, X and Spacer moieties is isotopically labeled; and salts and esters thereof.

Preferably at least one atom in at least one of the phosphate, X and Spacer moieties has the form of a stable heavy isotopic variant of the isotope that is most common in nature. Said isotope most common in nature is referred to herein as the ‘common isotope’.

A particularly preferred embodiment of the invention concerns a PTAG compound which is enriched in an isotopically labeled PTAG as defined in the foregoing, wherein at least one atom in at least one of the phosphate, X and Spacer moieties has the form of a stable heavy isotopic variant of the common isotope; and salts and esters thereof. In the context of this embodiment, enriched typically means that the abundance of a heavy isotopic variant at a selected position in the molecular structure is at least 5, at least 10, at least 25, at least 50, at least 100, at least 250, at least 500 or at least 1000 times higher than it is in nature. In a preferred embodiment a PTAG compound is provided wherein the respective heavy isotopic variant of the common isotope is at least 5, at least 10, at least 25, at least 50, at least 100, at least 250, at least 500 or at least 1000 times more abundant than any other isotopic variant at the selected position in the molecular structure.

In the above formula ‘X—’ represents a reactive group, which is reactive towards a chemical moiety on a target molecule in a sample and therefore can be covalently coupled to said target molecule, such as a protein, a peptide, a nucleic acid molecule, a lipid, a carbohydrate and/or a steroid, preferably a protein or a peptide. Suitable criteria for selection of a reactive group ‘X—’ include, but are not limited to, the type of molecule to be targeted with the PTAG reagent, the particular chemical moiety on the target molecule to be reacted, the stability of the reactive group under the buffer conditions used for the reaction, and the like. Any of these or other desirable criteria can be considered by one skilled in the art. Suitable reactive groups are well known to those skilled in the art (see, for example, Hermanson, supra, 1996; Glazer et al., Laboratory Techniques in Biochemistry and Molecular Biology: Chemical Modification of Proteins, Chapter 3, pp. 68-120, Elsevier Biomedical Press, New York (1975); Pierce Catalog, Pierce, Rockford Ill.). Any of a variety of reactive groups can be incorporated into a PTAG so long as the reactive group can be covalently coupled to a target molecule in a sample. Examples of particularly preferred target moieties for the PTAG are amino acids which are frequently used for selective labeling, such as cysteine, methionine and lysine. The reactive group X can also have specific reactivity towards peptides or proteins that have undergone selective protein derivatization. Examples are the selective targeting of N-terminal peptides after transamination reaction, tryptophan peptides after malondialdehyde derivatization, glycopeptides after oxidation of carbohydrates or arginine peptides after modification with vicinal dicarbonyl compounds.

For example, a PTAG can be coupled to a polypeptide via a thiol reactive group, which can react with free thiols of cysteine or reduced cystines in the polypeptide. Exemplary thiol reactive groups include halo acetylamino, maleimides, alkyl and aryl halides, haloacetyls, α-haloacyls, pyridyl disulfides, aziridines, acrylolyls, arylating agents, thiomethylsulfones, pyridylthio propionate, phenylmercury, phenyllead, phenylcadmium, thio sulfates, epoxides, nitrilles, disulfide exchange reagents, and the like. If desired, the polypeptides can be reduced, for example, with tri-butylphosphine, dithiothreitol, mercaptoethanol, and the like, prior to reacting with a PTAG, which is particularly useful when the PTAG contains a sulfhydryl reactive group.

A reactive group can also react with amines such as the α-amino group of a peptide or the ε-amino group of the side chain of Lys, for example, imidoesters, N-hydroxysuccinimidyl esters (NHS), sulfosuccinimidyl esters, isothiocyanates, isocyanates, acyl azides, sulfonyl halides, aldehydes, ketones, epoxides (oxiranes), carbonates, arylating agents, aryl halides, carbodiimides, anhydrides, tetrafluorophenyl esters, sulfodichlorophenol esters, dichlorotriazines, carboxylic acids, and the like, in the presence or absence of reducing agents such as sodium cyanogenborohydride. In addition, a vinyl sulfone such as R—SO₂—CH—CH₂ or R—NHSO₂—CH—CH₂, which can undergo a nucleophilic Michael-type addition reaction, can be used to react with sulfhydryl or amine groups on the target molecule.

A reactive group can also react with carboxyl groups found in Asp or Glu or the C-terminus of a peptide, for example hydrazines, hydroxylamines, primary aliphatic amines, primary aromatic amines, carbohydrazide, semicarbazides diazoalkanes, diazoacetyls, carbonyldiimidazole, carbodiimides, and the like, in the presence or absence of couplings reagents such as dicyclohexyl-carbodiimide or 2,3,5,6-tetrafluorphenyl trifluoroacetate.

A reactive group can also react with hydroxyl groups found in Ser, Thr and Tyr. An exemplary hydroxyl reactive group includes epoxide (oxiranes), carbonyldiimidazoles, N,N-disuccinimidyl carbonate, N-hydroxysuccinimidyl chlorofromate, dichlorotriazine, boronic acids, isocyanates (organic solvens), acyl nitriles (organic solvents), alkyl halogens, amine- or hydrazine reactive groups after (enzymatic oxidation), and the like.

A reactive group can also react with aldehyde or ketone groups found for example in glycosylated peptides. Exemplary aldehyde or ketone reactive groups include hydrazines, semicarbazides, carbohydrazide in the presence of absence of reducing agents such as sodium cyanogenborohydride, preferably hydroxylamines, primary aliphatic amines and primary aromatic amines

The reactive group can also be a group that reacts with a moiety on a target sample molecule in the presence of an extrinsic reagent such as a crosslinking reagent. For example, bis-maleimide can be used to crosslink an SH group on a target molecule with an SH group on the PTAG reagent. These and other cross-linking reagents suitable for coupling a peptide to a PTAG reagent are well know to those skilled in the art (see, for example, Hermanson, Bioconjugate Techniques, Academic Press, San Diego (1996); Glazer et al., supra, 1975). The reactive groups described above can form a covalent bond with the target sample molecule. However, it is understood that a PTAG reagent can contain a group that can non-covalently interact with a sample molecule so long as the interaction has high specificity and affinity.

In a preferred embodiment of the invention, an organophosphate compound of the above structure is provided, wherein X— is:
a thiol reactive group selected from iodoacetylamino, maleimido, pyridylthio propionate, chloroacetylamino, phenylmercury, phenyllead, phenylcadmium, thiosulfates, epoxides, nitrilles, aziridines, acryloyl, aryl halides, thiol-disulfide exchange reagents, preferably bromoacetylamino;
an amine reactive group selected from succinimidyl esters, sulfosuccinimidyl esters, tetrafluorophenyl esters, and sulfodichlorophenol esters, isothiocyanates, isocyanates, sulfonyl halides, dichlorotriazines, aryl halides, acyl azides, carboxylic acids, anhydride, epoxides, ketones, imidoesters, and carbodiimides;
a carboxylic acid reactive groups selected from hydrazines, hydroxylamines, primary aliphatic amines and primary aromatic amines, semicarbazides, carbohydrazide using carbodiimides;
a hydroxyl reactive group selected from epoxide, carbonyldiamidazole, N,N-disuccinimidyl carbonate, N-hydroxysuccinimidyl chlorofromate, dichlorotriazine, boronic acids, isocyanates, acyl nitriles, alkyl halogens; or
an aldehyde or ketone reactive group selected from hydrazines, semicarbazides, carbohydrazide.

In a particularly preferred embodiment of the invention affinity tags are provided that can be used in proteomics analysis, for which purpose cysteine residues are a very suitable target, such that X preferably represents a thiol reactive group as defined above.

The ‘spacer’ is used to bridge the phosphate moiety and the reactive group (X). The spacer may comprise a cleavable group. A cleavable group is particularly useful when mass spectrometry (MS) is being used as an analytical method. However, it is to be understood that cleavage of a cleavable group or the use of a cleavable group is not required in accordance with the present invention. Any of a number of chemical groups can be used to bridge the affinity tag with the reactive group. Suitable criteria for selection of a spacer include but are not limited to a desired size, solubility, chemical reactivity, ability to cleave or not cleave the moiety (by the presence of a cleavable linker), the type of cleavage method to be used, and the like. Any of these or other desirable criteria can be considered by one skilled in the art for designing a suitable PTAG according to the invention.

The spacer in combination with the reactive group X, has a sufficient length to allow the reactive group to bind to a target molecule and the phosphate group to bind to its cognate binding partner. From this perspective the spacer can be virtually any saturated or unsaturated, branched or linear, substituted or unsubstituted aliphatic, heteroaliphatic, aromatic or heteoaromatic chain having a length of 3 atoms or more. However, the main spacer chain preferably is relatively short, typically having a length of no more than 20 atoms. As a results, PTAG's are relatively small so that they can easily target specific sample molecules, show good solubility and undergo minor retention on reversed phase liquid chromatography. The spacer bridge can be equipped with a certain increased number of alkane building blocks, to provide some additional/different properties which can be useful in certain proteomics strategies, such as decreased reactivity, increased retention on reversed phase liquid chromatography and better adsorption or elution properties from solid phase resins such as IMAC and TiO2. As a general rule, it is preferred that the length of the main spacer chain is 3-10 atoms in case it does not contain any heteroatoms or 3-20 atoms in case it does contain one or more heteroatoms in the main chain or its substituents.

In an embodiment, the spacer is simply composed of a C3-C10 alkyl, alkenyl or alkynyl chain. In another embodiment the spacer is composed of a C3-C20 heteroalkyl, heteroakenyl or heteroalkynyl chain. In another embodiment of the invention said alkyl, alkenyl, alkynyl, heteroalkyl, heteroalkenyl or heteroalkynyl chain is intersperse by one or more aryl groups, especially aryl groups which are part of a (photo)cleavable linker, as will be explained in more detail below. Common cleavable linkers will also typically comprise one or more heteroatoms.

In the aformentioned alkyl, alkenyl, alkynyl, heteroalkyl, heteroalkenyl, heteroalkynyl chains and/or phenyl groups, one or more hydrogen atoms may be substituted by a suitable substituent, such as those selected from the group consisting of, —OR, ═O, ═NR, ═N—O—R, —NRR′, —SR, -halogen, —OC(O)R, —C(O)R, CO₂R, —CONRR′, —OC(O)NRR′, —NRC(O)R′, —NR—C(O)NR″, R′″, —NC(O)₂R, —NR—C(NRR″R′″)═NR″, —NR—C(NRR″)═NR′″, —SOR, —S(O)₂R, S(O)₂NRR″, —NRSO₂R′, —CN and —NO₂ wherein, R, R′, R″ and R′″ represent hydrogen or a branched or linear alkyl, alkenyl, alkynyl, alkoxy, thioalkoxy, heteroalkyl, cycloalkyl, cycloalkenyl, aryl, cycloalkylalkyl, cycloalkenylalkyl, or arylalkyl moiety, preferably hydrogen or a branched or linear alkyl, alkoxy, thioalkoxy, heteroalkyl, aryl or arylalkyl moiety.

As utilized in this document, the term “alkyl”, either alone or within other terms, means an acyclic alkyl radical. Lower alkyls are preferred, typically containing from 1 to 10, more preferably from 1 to about 8 carbon atoms and most preferably 1 to about 6 carbon atoms. Said alkyl radicals may be optionally substituted as defined elsewhere in this document. Examples of such radicals include methyl, ethyl, chloroethyl, hydroxyethyl, n-propyl, oxopropyl, isopropyl, n-butyl, cyanobutyl, isobutyl, sec-butyl, tert-butyl, pentyl, aminopentyl, iso-amyl, hexyl, octyl and the like.

The term “alkenyl” refers to an unsaturated, acyclic hydrocarbon radical in so much as it contains at least one double bond. Lower alkenyls are preferred, typically containing from 2 to 10 carbon atoms, preferably from 2 to 8 carbon atoms and most preferably 2 to about 6 carbon atoms. Said alkenyl radicals may be optionally substituted as defined elsewhere in this document. Examples of suitable alkenyl radicals include ethenyl, 1-propenyl, 2-propenyl, 2-methyl-1-propenyl, 1-butenyl, 2-butenyl and the like.

The term “alkynyl” refers to an unsaturated, acyclic hydrocarbon radical in so much as it contains one or more triple bonds. Lower alkynyls are preferred, typically containing from 2 to 10 carbon atoms, preferably having from 2 to 8 carbon atoms and most preferably from 2 to 6 carbon atoms. Said alkynyl radicals may be optionally substituted with groups as elsewhere in this document. Examples of suitable alkynyl radicals include ethynyl, propynyl, hydroxypropynyl, butyne-1-yl, butyn-2-yl, pentyne-1-yl, pentyne-2-yl, 4 methoxypentyn-2-yl, 3-methylbutyn-1-yl, hexyne-1-yl, hexyne-2-yl, hexyne-3-yl, 3,3-dimethylbutyn-1-yl radicals and the like.

The term “cycloalkyl” refers to carbocyclic radicals typically having 3 to 10 carbon atoms, preferably 3 to 8 carbon atoms, most preferably 5 to 8 carbon atoms. Said cycloalkyl radicals may be optionally substituted as defined elsewhere in this document. Examples of suitable cycloalkyl radicals include, but are not limited to, cyclopropyl, cyclobutyl, cyclopentyl, cyclohexyl and cycloheptyl.

The term “cycloalkenyl” embraces carbocyclic radicals having 3 to 10 carbon atoms and one or more carbon-carbon double bonds. Preferred cycloalkenyl radicals are “lower cycloalkenyl” radicals having 3-8 carbon atoms, more preferably 5-8. Examples include radicals such as cyclobutenyl, cyclopentenyl, cyclohexenyl and cycloheptenyl.

The term “aryl”, alone or in combination, means a 5-10 membered carbocyclic aromatic system containing one, two or three rings wherein such rings may be attached together in a pendant manner or may be fused. The term “fused” means that a second ring is present having two adjacent atoms in common with the first ring. The term “fused” is equivalent to the term “condensed”. The term “aryl” embraces aromatic radicals such as phenyl, naphthyl, tetrahydronaphthyl, indane and biphenyl.

The term “heteroaryl” (on its own or in any combination, such as “heteroaryloxy”, or “heteroaryl alkyl”) is used herein to mean a 5-10 membered aromatic ring system containing one, two or three rings, which may be attached in a pendant manner or may be fused, wherein at least one of said rings contains one or more heteroatoms selected from the group consisting of N, O or S. Examples include, but are not limited to, pyrrole, pyrazole, furan, thiophene, quinoline, isoquinoline, quinazolinyl, pyridine, pyrimidine, oxazole, thiazole, thiadiazole, tetrazole, triazole, imidazole, or benzimidazole.

The terms “cycloalkylalkyl”, “cycloalkenylalkyl”, “arylalkyl” and “heteroarylalkyl” embrace, respectively, the afore-defined cycloalkyl, cycloalkenyl, aryl and heteroaryl radicals attached to the main molecular moiety, i.e. the basic moiety depicted in the formulae, through an alkyl radical, typically a lower alkyl radical having 1-10, preferably 1-8, most preferably 1-6 carbon atoms, as will be understood by those skilled in the art. Representative examples of arylalkyl include, but not limited to, phenylmethyl, phenylethyl and naphthylmethyl. Representative examples of heteroarylalkyl groups include, but are not limited to, thiazolylmethyl, thienylmethyl, furylmethyl, imidazolylmethyl and pyridylmethyl.

In an embodiment of the invention an organophosphate compound is provided wherein the spacer is a linear alkyl chain, preferably a linear C₃-C₅ alkyl chain. In an embodiment of the invention an organophosphate compound as defined in the foregoing is provided wherein ‘SPACER’ may represent —C.C— instead of PTAG's of the present invention are compatible with mass spectrometry, meaning that there is no necessity to cleave off the phosphate affinity group prior to analysis. However, one must take into account that organophosphate tagged samples may show neutral loss of the phosphate group during MS/MS fragmentation. Solutions for this problem are available, e.g. by using MS3, pseudo MS3 or a new type of fragmentation technique called Electron Transfer Dissociation (ETD).

Another way to circumvent neutral loss of the phosphate group during MS/MS fragmentation is to chemically or enzymatically cleave-off the phosphate functional group prior to MS/MS analysis. By removing the phosphate group, the detection and fragmentation performance may be further enhanced. Hence, in a preferred embodiment of the invention the Spacer comprises a cleavable linker.

A cleavable linker can typically be a photocleavable linker or chemically cleavable linker.

Suitable photocleavable linkers include for example linkers containing o-nitrobenzyl, desyl, trans-o-cinnamoyl, m-nitrophenyl, benzylsulfonyl groups (see, for example, Dorman and Prestwich, Trends Biotech. 18:64-77 (2000); Greene and Wuts, Protective Groups in Organic Synthesis, 2nd ed., John Wiley & Sons, New York (1991); U.S. Pat. Nos. 5,143,854; 5,986,076; 5,917,016; 5,489,678; 5,405,783). One skilled in the art will readily recognize that these exemplary photocleavable groups as well as others can be used in a PTAG reagent.

A chemically cleavable linker can also be used. Exemplary chemically cleavable linkers can contain a disulfide, which can be cleaved with reducing agents; a diol, which can be cleaved with periodate; a diazo bond, which can be cleaved with dithionate; an ester, which can be cleaved with hydroxylamine; a sulfone, which can be cleaved with base (see Hermanson, Bioconjugate Techniques, Academic Press, San Diego (1996); Pierce Chemical Co., Rockford Ill.). Other common examples include —S—S—, diarylmethyl or trimethylarylmethyl groups, silyl esters, carbamates, oxyesters, thioesters, thionoesters, and fluorinated amines.

A thermally cleavable linker can also be employed in accordance with the present invention, such as a linker consisting of one or more nucleic acid bases.

Furthermore, a linker cleavable by an enzyme can be used. For example, a protease can be used to cleave a cleavable linker having a suitable recognition sequence for the protease. Hence in an embodiment an organophosphate compound as defined herein before is provided, wherein the spacer comprises a peptide chain of at least 3, more preferably at least 4, most preferably at least 5 consecutive amino acids, which constitute of include a protease recognition sequence.

Particularly useful proteases are endopeptidases such as factor Xa, tobacco etch virus (TEV) protease, trypsin, chymotrypsin, Staphylococcus aureus protease, submaxillaris protease, and the like. The protease can be selected based on the incorporation of a particular cleavable recognition sequence into the linker. Other considerations for selecting a protease include the presence or absence of a recognition sequence in the molecule being captured and released. For example, a rare cleaving protease such as TEV protease or factor Xa can be used to cleave a functional group containing the corresponding protease recognition sequence, resulting in release of the captured molecule. Such rare cleaving proteases are particularly useful for releasing an intact polypeptide molecule since the recognition sequence for these proteases would not occur in the vast majority of polypeptides. Alternatively, a polypeptide sample can be treated with a specific protease, and the digested peptides isolated by the methods disclosed herein. In such a case, the captured peptides would not contain a recognition sequence for the protease used for cleavage since the polypeptide has already been digested. In addition, if desired, an intact polypeptide can be captured and digested with a protease after binding to the solid support, resulting in the incorporation and release of a label on the peptide fragment of the polypeptide that was captured on the solid support. Thus, protease digestion can be used before or after capture of a sample molecule, as desired.

A cleavable functional group can also be a recognition sequence for an (endo)nuclease such as a restriction enzyme. Thus, an appropriate recognition sequence for a restriction enzyme can be incorporated as a cleavable functional group and cleaved with the respective restriction enzyme. It is understood that such a nucleotide functional group can be useful for capturing and releasing a nucleic acid or a polypeptide, or any other type of molecule, as desired. Similarly, a protease recognition sequence can be useful for capturing and releasing a polypeptide, nucleic acid or any other type of molecule, as desired.

Other enzyme cleavable linkers may comprise a glycosidase sensitive glycosidic linkage; a β-lactamase-sensitive P-lactam analog, a thrombin cleavage sequence or an enterokinase cleavage sequence.

Hence, in a preferred embodiment of the invention an organophosphate compound as defined above is provided, wherein said Spacer is a cleavable linker, selected from the group consisting of:
photo cleavable groups, preferably O-nitrobenzyl, desyl, trans-o-cinnamoyl, m-nitrophenyl, benzylsulfonyl groups
thermally cleavable groups, preferably a moiety comprising one or multiple nucleic acid bases;
chemically cleavable linkers, preferably diols, diazo groups, esters, sulfones, —S—S—, diarylmethyl or trimethylarylmethyl groups, silyl esters, carbamates, oxyesters, thioesters, thionoesters, fluorinated amines and esters, diols;
enzyme-cleavable linkers, preferably protease-sensitive amides or esters, P-lactamase-sensitive P-lactam analogs, thrombin cleavage sequences, enterokinase cleavage sequences; glycosidase-cleavable sugars, or nuclease cleavable phosphodiesters.

When the spacer comprises a cleavable linker, it is preferred that the portion of the PTAG which will remain attached to a sample molecule upon cleavage of a cleavable linker has sufficient chemical characteristics to allow the incorporation of one or more isotopic atoms.

A suitable example of a PTAG for selective labeling of sulfurhydryl groups (SH), such as cysteine, includes 3-[(bromoacetyl)amino]propyl dihydrogen phosphate. Amino groups, such as α-amines at protein/peptide N-termini as well as ε-amines at lysines, react rapidly with active esters and aldehyde functional groups with or without the addition NaBH₄ or NaCNBH₃. Examples of such PTAG's with a reactive ester or aldehyde functionality include 4-[(2,5-dioxopyrrolidin-1-yl)oxy]-4-oxobutyl dihydrogen phosphate and 4-oxobutyl dihydrogen phosphate. For tagging aldehydes, ketones and carboxylates groups, PTAG's holding an oxime or amine reactive group are envisaged, such as 3-(aminooxy)propyl dihydrogen phosphate and 3-aminopropyl dihydrogen phosphate. These PTAG's could be used, for example, to selectively tag carbohydrates after treating first with periodate to generate an aldehyde or keton and is used with or without NaBH4 and NaCNBH3. Another example is the selective coupling of PTAG's to α-amines of protein/peptide N-termini after a transamination reaction to generate a keton. Carboxylic acids are reactive to these PTAG in the presence of coupling reagents such as dicyclohexyl-carbodiimide. Finally, tryptophan containing peptides or proteins could be selectively tagged with after malondialdehyde derivatization. Hence, in a particularly preferred embodiment of the invention, an organophosphate selected from the group consisting of 3-[(bromoacetyl)amino]propyl dihydrogen phosphate (1); 3-aminopropyl dihydrogen phosphate (2); 4-oxobutyl dihydrogen phosphate (3); 3-(aminooxy)propyl dihydrogen phosphate (4); 4-[(2,5-dioxopyrrolidin-1-yl)oxy]-4-oxobutyl dihydrogen phosphate (5); and salts and esters thereof is provided.

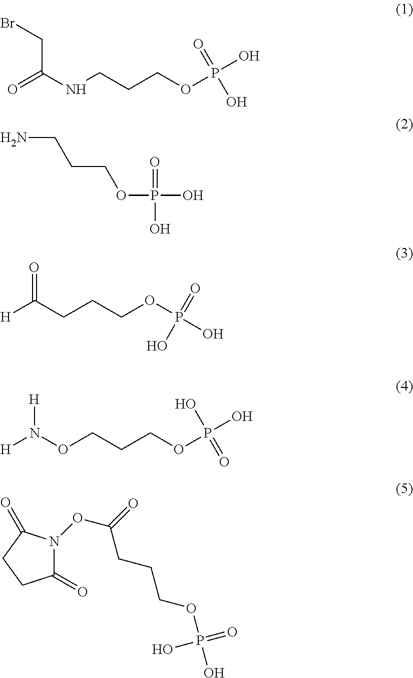 [SMILES: O=C(CBr)NCCCOP(=O)(O)O ; NCCCOP(=O)(O)O ; O=CCCCOP(=O)(O)O ; NOCCCOP(=O)(O)O ; O=C(CCCOP(=O)(O)O)ON1C(=O)CCC1=O]

In an embodiment of the invention, the spacer moiety may comprise, as a substituent, an optically detectable moiety, such as a fluorophore or a chromorphore.

Such an optically detectable moiety allows a PTAG-tagged molecule to be visualized, which may be convenient, especially, in the process of separating PTAG-bound target molecules from non-bound molecules. Fluorophores suitable for this purpose include, for example rhodamine-type fluorophores, e.g. tetramethylrhodamine, rhodamine B, rhodamine 6G, sulforhodamine B, Texas Red (sulforhodamine 101), rhodamine 110, tetramethylrhodamine-5-(or 6), lissamine rhodamine B, and the like. Other suitable fluorophores include 7-nitrobenz-2-oxa-1,3-diazole (NBD); fluorescein as well as derivatives thereof; napthalenes such as dansyl (5-dimethylaminonapthalene-1-sulfonyl); coumarin derivatives, such as 7-amino-4-methylcoumarin-3-acetic acid (AMCA), 7-diethylamino-3-[(4′-(iodoacetyl)amino)phenyl]-4-methylcoumarin (DCIA), Alexa fluor dyes (Molecular Probes), and the like; 4,4-difluoro-4-bora-3a,4a-diaza-s-indacene (BODIPY™) as well as derivatives thereof (Molecular Probes; Eugene Oreg.); pyrenes and sulfonated pyrenes such as Cascade Blue™ as well as derivatives thereof, including 8-methoxypyrene-1,3,6-trisulfonic acid; pyridyloxazole derivatives; dapoxyl derivatives; Lucifer Yellow (3,6-disulfonate-4-amino-naphthalimide) as well as derivatives thereof; and CyDye™ fluorescent dyes (Amersham Pharmacia Biotech; Piscataway N.J.). Exemplary chromophores include phenolphthalein, malachite green, nitroaromatics, such as nitrophenyl; diazo dyes, dabsyl (4-dimethylaminoazobenzene-4′-sulfonyl), and the like (see Hermanson, 1996).

Such an optically detectable moiety, if present, is preferably separated from the reactive group X by a cleavable linker, such as those described herein before, such that it can be cleaved before mass spectrometric analysis of the PTAG tagged components.

In an embodiment of the invention, the spacer may comprise, as a substituent, one or two additional phosphate groups. It has been found that PTAG compounds according to this embodiment exhibit increased affinity to capture agents such as TiO₂. As will be understood by those skilled in the art, such increased binding affinity may allow for improved and/or more efficient separation of tagged and non tagged molecules. In a preferred embodiment one additional phosphate group is present. In another preferred embodiment of the invention the Spacer moeity does not comprise an (additional) phosphate group.

The organophosphate compound of the invention typically comprises one or more stable isotope tag. As used herein the term “stable isotope tag” refers to a chemical moiety having suitable chemical properties for incorporation of a stable isotope, allowing the generation of differentially labeled PTAG's which can be used to differentially tag a target substance in two samples. As explained before differentially labeled PTAG's are chemically identical which means that they behave identically during reversed phase chromatography and therefore elute simultaneously. However, the variants differ in mass due to their unique stable isotope pattern of the affinity labels and therefore allow relative quantification of protein concentrations by measurements of the peak areas.

In a particularly preferred embodiment of the invention an organophosphate compound as defined herein before is provided, wherein at least one of the reactive groups X and the Spacer moiety comprise an isotope tag. These organophosphate compounds allow for quantification based on stable isotope patterns, even when the phosphate group is removed or lost.

In an even more preferred embodiment, an organophosphate compound is provided wherein the spacer comprises a cleavable linker moiety separating the phosphate moiety, a portion of the spacer connected directly to said phosphate moiety and the optional optically detectable moiety from the reactive group X and a portion of the spacer connected directly to X, wherein at least X or said portion of the spacer connected directly to X contains an isotope tag.

Such a structure is particularly useful with a view to applying mass spectrometry based analytical methods, where it may be advantageous to be able to release the phosphate group and the optional optically detectable moiety while retaining a portion of the PTAG containing one or more isotopic labels.

The present invention thus also provides organophosphate compounds as defined herein before, wherein at least one atom in at least one of the X and Spacer moieties is a stable isotope. In an even more preferred embodiment, an organophosphate compound is provided wherein the spacer comprises a cleavable linker separating the phosphate moiety and the optional optically detectable moiety from the reactive group X and a portion of the spacer, wherein at least one atom in at least one of X and the portion of the spacer is a stable isotope.

The present invention thus also provides organophosphate compounds as defined herein before, wherein at least one of the X and Spacer moieties is isotopically labeled, meaning that at least one of said moieties comprises a stable heavy isotopic variant of the common isotope. In an even more preferred embodiment, an organophosphate compound is provided wherein the spacer comprises a cleavable linker separating the phosphate moiety and the optional optically detectable moiety from the reactive group X and a portion of the spacer, wherein at least one atom in at least one of X and the portion of the spacer is isotopically labeled, meaning that at least one atom in said moieties is a stable heavy isotopic variant of the common isotope.

A particularly useful stable isotope pair is hydrogen and deuterium, which can be readily distinguished using mass spectrometry as light and heavy forms, respectively. Any of a number of isotopic atoms can be incorporated into the PTAG so long as the heavy and light forms can be distinguished using mass spectrometry, for example, ²H, ¹³C, ¹⁵N, ¹⁷O, ¹⁸O, 32P, ³³P and ³⁴S. Differential isotopic tags will generally differ by minimally 2 mass units and up to about 20 mass units, preferably 4-16 mass units, more preferably 6-12 mass units, and the like. One skilled in the art can readily determine an appropriate mass differential suitable for isotope tags for detecting mass differences of PTAG-labeled molecules using mass spectrometry. Multiple isotopes can be incorporated. In a particularly preferred embodiment of the invention an organophosphate compound as defined herein before is provided comprising at least one, at least two, at least three or at least four stable isotopes selected from the group consisting of ²H, ¹³C, ¹⁵N, ¹⁷O, ¹⁸O, ³²P, ³³P and ³⁴S, preferably from the group consisting of ²H and ¹³C. Particularly preferred examples of isotopically labeled organophosphate compounds according to the invention include those selected from the group of 3-[(bromoacetyl)amino](²H₆)propyl dihydrogen phosphate (6); 3-amino(²H₆)propyl dihydrogen phosphate (7); 4-oxo(1,1,2,2-²H₄)butyl dihydrogen phosphate (8); 3-(aminooxy)(²H₆)propyl dihydrogen phosphate (9); and 4-[(2,5-dioxopyrrolidin-1-yl)oxy]-4-oxo(²H₆)butyl dihydrogen phosphate (10); and salts and esters thereof.

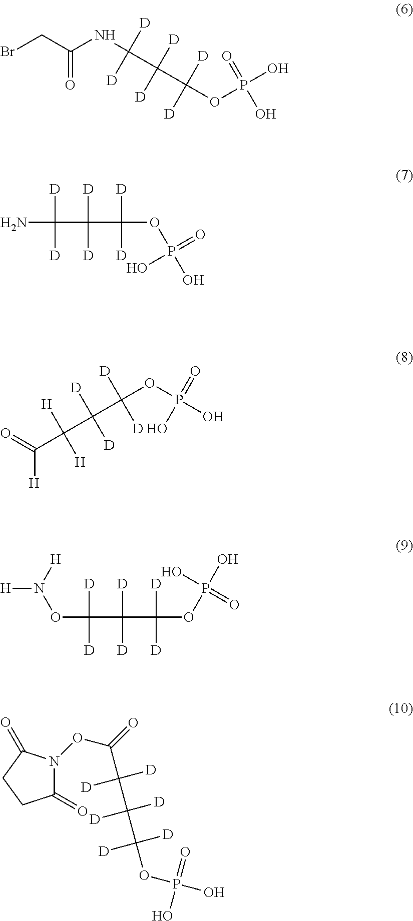 [SMILES: [2H]C([2H])(NC(=O)CBr)C([2H])([2H])C([2H])([2H])OP(=O)(O)O ; [2H]C([2H])(N)C([2H])([2H])C([2H])([2H])OP(=O)(O)O ; [2H]C([2H])(CC=O)C([2H])([2H])OP(=O)(O)O ; [2H]C([2H])(ON)C([2H])([2H])C([2H])([2H])OP(=O)(O)O ; [2H]C([2H])(OP(=O)(O)O)C([2H])([2H])C([2H])([2H])C(=O)ON1C(=O)CCC1=O]

The invention also relates to salt analogous of the organophosphate compounds, such as the soldium salts. A particular advantage of using these and other salts is that the stability of the product in the solid form is significantly improved.

The PTAG reagents of the invention can be used to label target molecules in a sample. The methods are particularly useful for labeling polypeptides in a sample, and can thus be used for qualitative and quantitative proteome analysis. Although labeling of polypeptides is exemplified herein, it is understood that sample molecule having chemical properties reactive with the PTAG reagent reactive group can be labeled with a PTAG reagent of the invention. For example, a PTAG reagent can be used to label nucleic acids, carbohydrates, lipids, metabolites or other sample molecules. The invention also provides a method of isolating a polypeptide in a sample. The method can include the steps of contacting a sample with a PTAG reagent of the invention under conditions allowing the reactive group to bind to and react with one or more polypeptides in the sample, thereby tagging one or more target molecules with the reagent; resolving the polypeptides in the sample; contacting the tagged molecules with a capture moiety; and isolating the tagged target molecules.

Hence, another aspect of the present invention concerns a method of enriching for one or more target molecules in a sample, said method comprising the steps of:
a) providing said sample comprising said one or more target molecules;
b) combining the sample with a composition comprising an organophosphate compound as defined herein before;
c) subjecting the sample to conditions that allow for selective binding of the reactive group ‘X’ of the organophosphate compound to the one or more target molecules;
d) separating organophosphate compound bound substances from unbound substances, using a separation technique based on specific chemical and/or physical properties of the phosphate group, yielding a fraction enriched in the one or more target molecules.

As used herein, the term “sample” typically refers to a biological fluid, cell, tissue, organ or portion thereof, that includes one or more different molecules such as nucleic acids, polypeptides, or small molecules. A sample can be a tissue section obtained by biopsy, or cells that are placed in or adapted to tissue culture. A sample can also be a biological fluid specimen such as blood, urine or saliva. A sample can additionally be a cell extract from any species, including prokaryotic and eukaryotic cells as well as viruses. A tissue sample can be further fractionated, if desired, to a fraction containing particular cell types.

In an embodiment of the invention the sample is subjected to a pretreatment step wherein target molecules are modified. For example, in the case of proteomics analysis, it is often useful to digest proteins and polypeptide into smaller fragments, for example, by enzymatic cleavage with one or more proteases, such as trypsin, chymotrypsin, pepsin, papain, Staphylococcus aureus (V8) protease, and the like, or chemically, for example, using CNBr, acid or other chemical reagents.

One skilled in the art can readily determine appropriate conditions so that the reactive group of the PTAG can react with a target molecule, including appropriate buffers, salts, pH, temperature, and the like (see Hermanson, 1996).

The PTAG-labeled target molecules can be separated from non labeled molecules relying on chemical and/or physical properties of the phosphate group, for example the specific and reversible binding thereof to a ‘capture agent’ such as metal oxides or antibodies, attached to a solid support such as a bead, resin, membrane or the like. In a preferred embodiment of the invention a method as defined above is provided, wherein the separation is based on the principle of:
capture of the phosphate groups to solid phase bound metal oxides, such as titanium dioxide, zirconium dioxide, titanium, aluminum oxide, niobium oxide, aluminum hydroxide, alumina, gallium oxide, tin dioxide;
ion exchange, such as cation exchange, anion exchange and mixed anion cation exchange;
immobilized affinity chromatography, such as Fe-IMAC, Al-IMAC, Ga-IMAC, Co-IMAC, Zr-IMAC, Ti-IMAC;
hydrophillic interaction chromatography;
calcium phosphate precipitation;
capture of the phosphate group by anti-phosphotyrosine antibodies, anti-phosphoserine antibodies, anti-phosphothreonine antibodies, which are typically bound to a solid support;
phosphoramidate chemistry; and/or
isoelectric focussing.

One skilled in the art can readily determine a desired modality and determine proper conditions to achieve optimal results. The use of metal oxides is particularly preferred in accordance with the present invention.

In a preferred embodiment of the invention, the method includes the step of releasing the phosphate moiety from the PTAG-labeled target molecule following the separation organophosphate compound bound substances from unbound substances, wherein a portion of the PTAG containing a stable isotope is retained. Hence, in a preferred embodiment of the invention a method as defined in any of the foregoing is provided, which further comprises the step of subjecting the sample to conditions resulting in cleavage of the phosphate moiety from the target molecule following step d). One skilled in the art can readily determine appropriate conditions so that the reactive group of the PTAG reagent can react with a target molecule, including appropriate buffers, salts, pH, temperature, and the like

The reagents and/or conditions for achieving the release of the phosphate group will depend, in particular on the presence and identity of a moiety defined herein before as the cleavable linker. In an embodiment of the invention however the phosphate moiety can be released regardless of the presence of a cleavable linker, by hydrolysis of the phosphate group. Hence, a method is also provided as defined herein before, wherein the phosphate moiety is hydrolyzed using specific phosphatase enzymes or acid or base catalyzed hydrolysis conditions.

Furthermore, the release of the organophosphate group can be performed while the PTAG bound target molecule are captured on a solid phase, such that the steps of separating bound from unbound sample molecules and the release of the phosphate group are coupled in a particularly efficient manner. Hence, an embodiment of the invention provides a method as defined herein before, comprising the step of cleaving off of the phosphate group in order to release the tagged target molecule form a solid support containing a capture agent to which the phosphate moiety is bound.

The isolated PTAG-labeled molecules can be further characterized, if desired. A particularly useful method for characterizing sample molecules is mass spectrometry (MS), which can be used to identify and/or quantify the PTAG-labeled molecule. Thus, the invention provides a method of quantitative or qualitative determination of one or more target molecules in a sample is provided comprising an enrichment procedure as defined in any of the foregoing followed by subjecting the enriched sample to mass spectrometric analysis.

A variety of mass spectrometry systems can be employed in the methods of the invention for identifying and/or quantifying a sample molecule such as a polypeptide. Suitable mass analyzers with include, but are not limited to, matrix-assisted laser desorption time-of-flight (MALDI-TOF) mass spectrometers, electrospray ionization time-of-flight (ESI-TOF) mass spectrometers and Fourier transform ion cyclotron mass analyzers (FT-ICR-MS). Other modes of MS include ion trap and triple quadrupole mass spectrometers. Methods of mass spectrometry analysis are well known to those skilled in the art (see, for example, Yates, J. Mass Spect. 33:1-19 (1998); Kinter and Sherman, Protein Sequencing and Identification Using Tandem Mass Spectrometry, John Wiley & Sons, New York (2000); Aebersold and Goodlett, Chem. Rev. 101:269-295 (2001); Griffin et al., Curr. Opin. Biotechnol. 12:607-612 (2001)).

As explained herein before, differentially labeled isotope tags can be incorporated into the PTAG. The differential tags can be reacted with a test and control sample and run together or in parallel. The differentially labeled molecules can be analyzed using mass spectrometry (MS). Because the sample molecules are differentially labeled with light and heavy isotopes, the peptide fragments can be distinguished by MS. In this way it is possible to analyze side-by-side several proteome samples, e.g. a test sample and a control sample or a test sample and an internal standard, by respectively modifying with PTAG having distinct isotopic labels. Subsequently, (relative) protein quantities can be determined between samples according the unique isotope pattern. Using an internal standard it is possible to determine quantities of a target molecule can in a test sample. Such an internal standard is added in an amount within the quantitative range of the detection method. Hence, the methods of the invention can be used advantageously for the detection and accurate absolute quantification of particular peptides or proteins in a complex sample. The methods can also advantageously be used for the detection of changes or deviations in abundance of specific proteins in complex samples.

The methods of the invention are also applicable to the analysis of multiple sample polypeptides. For example, PTAG-labeled standard peptides can be added to identify and/or quantify two different polypeptides in a sample. In such a case, a particular PTAG reagent can be used to label sample polypeptides, and the corresponding standard peptides for the two polypeptides are labeled with an isotopically distinct version of the same PTAG reagent as used to label the sample polypeptides. The methods are advantageous in that multiple polypeptides in a sample can be analyzed. Any number of different polypeptides in a sample can be analyzed so long as there is sufficient resolution of the standard PTAG-labeled peptides for further analysis. Thus, the methods of the invention can be used to identify and/or quantify three or more polypeptides in a sample, four or more polypeptides, five or more polypeptides, six or more polypeptides, seven or more polypeptides, eight or more polypeptides, nine or more polypeptides, ten or more polypeptides, twelve or more polypeptides, fifteen or more polypeptides, or even greater numbers so long as the PTAG-labeled peptides can be sufficiently resolved for further analysis. Corresponding PTAG-labeled standards for the multiple polypeptides to be analyzed can be synthesized and added to the sample.

When analyzing multiple polypeptides in a sample, a single PTAG reagent can be used to label sample polypeptides. In addition, more than one type of PTAG reagent can be used to label sample polypeptides. For example, one type of PTAG reagent can be used to label reactive sulfhydryl groups of sample polypeptides and a second type of PTAG reagent can be used to label reactive amine groups. The use of PTAG reagents having different reactive groups to label the same sample can be used to distinguish between two polypeptides.

Another aspect of the invention concerns a kit of parts that comprising the isotopically distinct PTAG's of the invention, which is suitable in particular for use in the above described proteomics analyses. Hence, in an embodiment a kit of parts is provide comprising a first holder and a second holder, said first holder containing a quantity of a first organosphosphate compound as defined herein before; and said second holder containing a quantity of an organophosphate compound that is chemically identical but isotopically different from said first organophosphate compound. In a particularly preferred embodiment of the invention a kit is provided, wherein said second container holds a corresponding organophosphate compound which is not isotopically labeled. Furthermore, it is particularly preferred that the mass difference between said first and said second organophosphate compounds is 2-20 mass units, preferably 4-16 mass units, more preferably 6-12 mass units. The kit of the invention can typically further comprise one or more additional holders containing additional chemically identical but isotopically distinct organophosphate compounds; one or more additional holders containing solid phase bound capture agents for selective binding of phosphates; one or more holder containing reagents for calcium phosphate precipitation; one or more holders containing reagents involved in phosphoramidate chemistry; one or more additional holders containing reagents suitable for releasing the phosphate moiety from the capture agent; one or more holders containing reagents for cleavage of a cleavable linker; one or more holders containing reagents for hydrolysis of the phosphate moiety; and/or instructions for use of the kit.

Another aspect of the present invention concerns use of an organophosphate compound as defined in any one of the foregoing for labeling a target molecule selected from the group consisting of nucleic acids, carbohydrates, proteins, peptides, lipids and steroids, contained in a sample. As will be clear from the foregoing, a particularly preferred embodiment concerns such use for labeling a protein or peptide contained in a sample, preferably a biological sample. Most preferably the use of an organophosphate compound as defined in any one of the foregoing is provided for labeling a peptide or protein for proteomics analysis.

Yet another aspect of the invention concerns the intermediate products obtained in the methods of the present invention, notably a peptide or protein comprising, within its amino acid sequence and/or at its N-terminal and/or C-terminal positions, one or more building blocks represented by the formula:

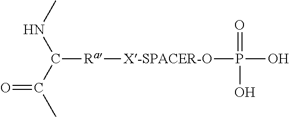

wherein

R^{a′} represents the residue of an amino acid side chain, preferably an amino acid side chain selected from, alanine, glycine, aspartic acid, glutamic acid, aspargine, glutamine, histidine, arginine, lysine, tryptophan, tyrosine, phynylalanine, methiodine, isoleucine, leucine, valine, praline, threonine, cysteine, serine, and glycosylated, or other post translationally modified forms and unnatural or chemical modified forms thereof;

—X′— represents the residue of a reactive group X; and
X and -Spacer- have the same meaning as defined herein before in relation to the structure of the organophosphate compound.

As used herein, the term residue means the part of the amino acid side chain and reactive group X, respectively, that remain after they have reacted to form the building block represented by the above depicted formula, as will be understood by those skilled in the art.

In a preferred embodiment of the invention, such a peptide or protein is provided wherein at least one atom in at least one of the —O—PO₃H₂, Y and Spacer moieties is a stable isotope, more preferably at least one of Y and the spacer moieties comprises a stable isotope.

In a preferred embodiment of the invention, such a peptide or protein is provided wherein at least one of the —O—PO₃H₂, Y and Spacer moieties, more preferably at least one of Y and the spacer moieties, is isotopically labeled, meaning that at least one of said moeities comprises a stable heavy isotopic variant of the common isotope.

Preferably said stable isotope is selected from the group consisting of ²H, ¹³C, ¹⁵N, ³²P, ³³P, ³⁴S and/or ¹⁸O.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The invention will be illustrated in more detail in the following examples, which are not intended to limit the scope here of in any way.

### EXAMPLES

The synthesis reactions to prepare 3-[(bromoacetyl)amino]propyl dihydrogen phosphate, 3-amino propyl dihydrogen phosphate, 3-(aminooxy)propyl dihydrogen phosphate, 4-oxobutyl dihydrogen phosphate and 4-[(2,5-dioxopyrrolidin-1-yl)oxy]-4-oxobutyl dihydrogen phosphate are described below. The deuterium PTAG analogous were prepared in a similar way.

### Example 1

### Synthesis of 3-[(bromoacetyl)amino]propyl dihydrogen phosphate

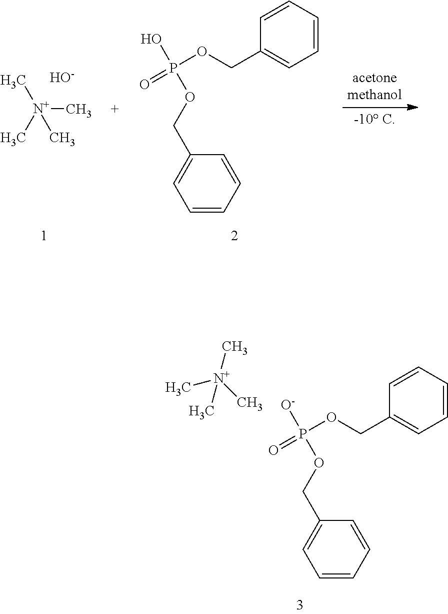

To a freshly prepared solution of dibenzylphosphate/acetone (2), a tetramethylammonium hydroxide/methanol/water solution was added dropwise at −10° C. Solvents were evaporated under a nitrogen stream.

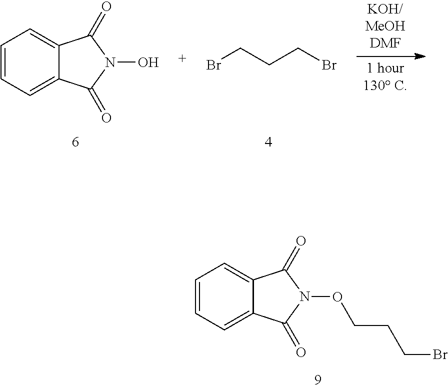 [SMILES: O=C1c2ccccc2C(=O)N1O ; BrCCCBr ; O=C1c2ccccc2C(=O)N1OCCCBr]

Potassium hydroxide was dissolved in methanol and N-hydroxyphthalimide (6) was added. The solution was mixed with a solution of 1,3 dibromopropane (4) in dimethylformamide (DMF) and heated to 130° C. for 1 hour under continuous stirring. After the addition of diethylether the reaction mixture was washed with 1M HCL and subsequently water. Volatiles were removed and the resulting crude product 2-(3-bromopropoxy)-1H-isoindool-1,3(2H)-dione (9) was purified on silica gel.

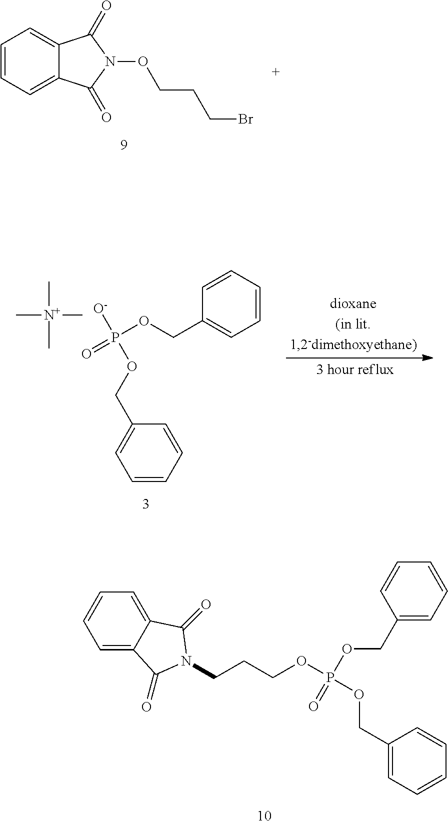

Tetramethylammonium dibenzylphosphate (3) was mixed with a 2-(3-bromopropoxy)-1H-isoindool-1,3(2H)-dione (9) in dioxane solution and refluxed for 3 hours. After the addition of diethylether, the reaction mixture was washed with water. Volatiles were removed and the resulting crude product 2-(3-dibenzylphosphatepropoxy)-1H-isoindool-1,3(2H)-dione (10) was purified on silica

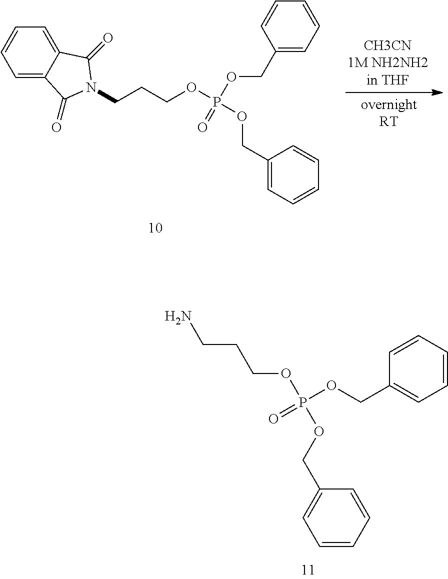

The 2-(3-dibenzylphosphatepropoxy)-1H-isoindool-1,3(2H)-dione (10) was dissolved in acetonitrille and 1 M hydrazine in THF was added. The mixture was stirred at room temperature overnight. The precipitate was filtered off and further washed with acetonitrille. The filtrate and acetonitrille wash fraction were combined and evaporated till dryness. The crude product 3-aminopropyl dibenzyl phosphate (11) has been obtained.

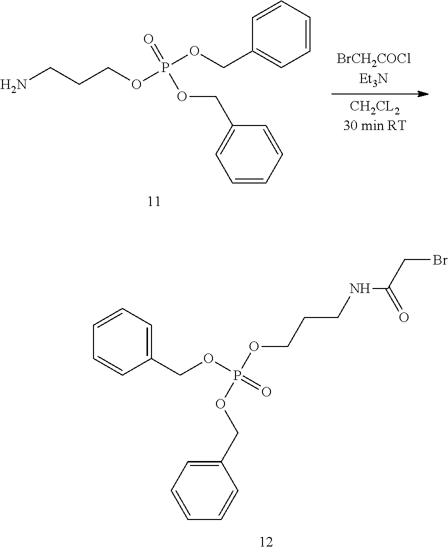 [SMILES: NCCCOP(=O)(OCc1ccccc1)OCc1ccccc1 ; O=C(CBr)NCCCOP(=O)(OCc1ccccc1)OCc1ccccc1]

The 3-aminopropyl dibenzyl phosphate (11), triethylamine and bromoacetyylchloride were mixed in dichloromethane and stirred at room temperature for 0.5 hour. Volatiles were removed and the resulting crude product dibenzyl 3-[(bromoacetyl)amino]propyl phosphate (12) was purified on silica gel.

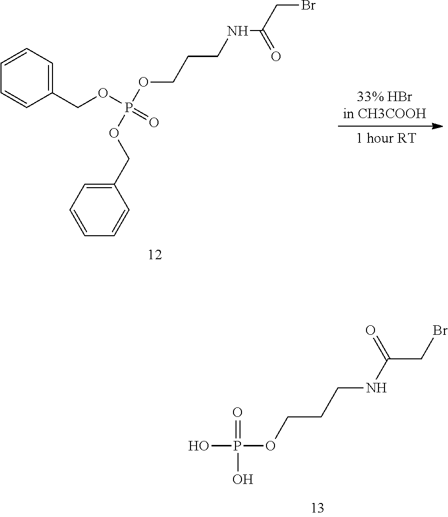 [SMILES: O=C(CBr)NCCCOP(=O)(OCc1ccccc1)OCc1ccccc1 ; O=C(CBr)NCCCOP(=O)(O)O]

The purified dibenzyl 3-[(bromoacetyl)amino]propyl phosphate (12) was fortified with 33% HBr in acetic acid. After 1 hour incubation at room temperature, the solvents were evaporated under a nitrogen stream. The 3-[(bromoacetyl)amino]propyl dihydrogen phosphate (13) has been obtained, and verified by ¹H-NMR (FIG. 1).

### Example 2

### Synthesis of 3-amino propyl dihydrogen phosphate

The intermediate product 2-(3-dibenzylphosphatepropoxy)-1H-isoindool-1,3(2H)-dione of the synthesis route of 3-[(bromoacetyl)amino]propyl dihydrogen phosphate (as described above), was used as starting product for the preparation of 3-amino propyl dihydrogen phosphate.

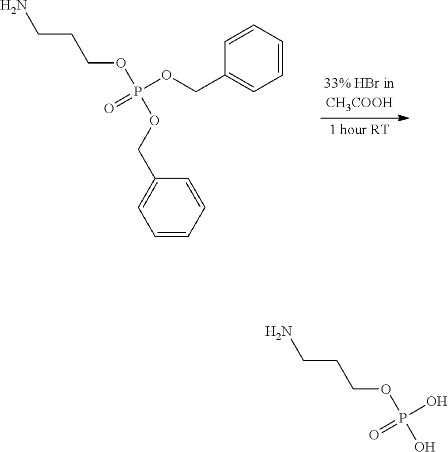 [SMILES: NCCCOP(=O)(OCc1ccccc1)OCc1ccccc1 ; NCCCOP(=O)(O)O]

The purified 2-(3-dibenzylphosphatepropoxy)-1H-isoindool-1,3(2H)-dione (1) was fortified with 33% HBr in acetic acid. After 1 hour incubation at room temperature, the solvents were evaporated by a nitrogen stream. The 3-[(bromoacetyl)amino]propyl dihydrogen phosphate (13) was obtained.

### Example 3

### Synthesis of 3-(aminooxy)propyl dihydrogen phosphate

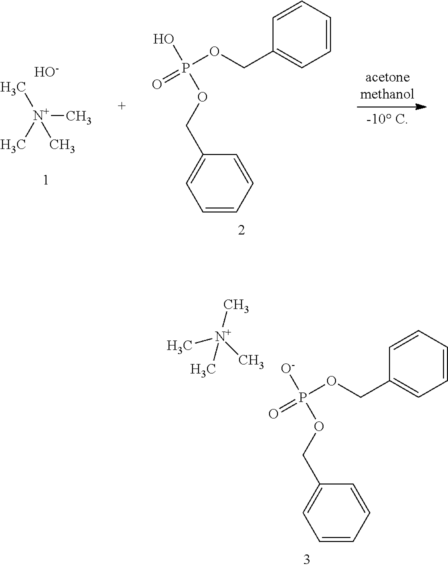

To a freshly prepared mixture of dibenzylphosphate/aceton (2), a mixture of tetramethylammonium hydroxide/methonal/water (1) was added dropwise at −10° C. Solvents were evaporated under a nitrogen stream.

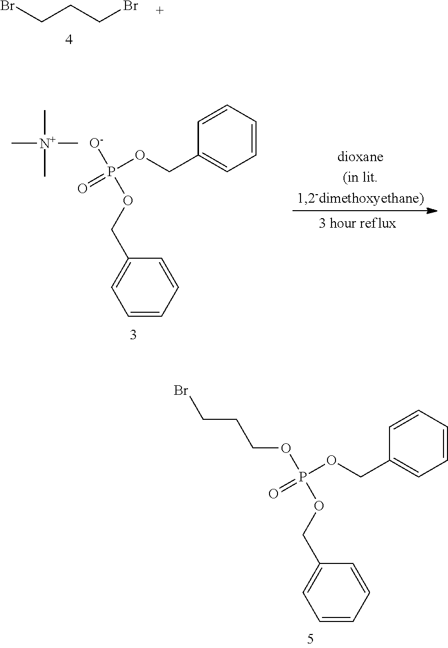

The tetramethylammonium dibenzylphosphate (3) was mixed with a solution of 1,3 dibromopropane (4) in dioxane and refluxed for 3 hours. The precipitated salt was filtered off and the filtrate was evaporated till dryness. The product dibenzyl 3-bromopropyl phosphate (5) was purified on silica gel.

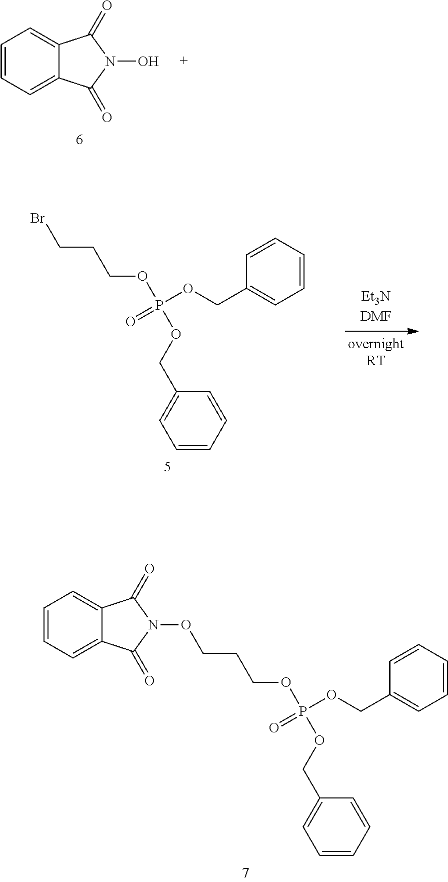 [SMILES: O=C1c2ccccc2C(=O)N1O ; O=P(OCCCBr)(OCc1ccccc1)OCc1ccccc1 ; O=C1c2ccccc2C(=O)N1OCCCOP(=O)(OCc1ccccc1)OCc1ccccc1]

A mixture of N-hydroxyphthalimide (6), dibenzyl 3-bromopropyl phosphate (5), triethylamine and DMF was prepared and stirred at room temperature overnight. After the addition of diethylether, the reaction mixture was washed with 1 M HCL and subsequently water, and further dried using Mg SO₄. Volatiles were removed and the resulting crude product (7) was purified on silica gel.

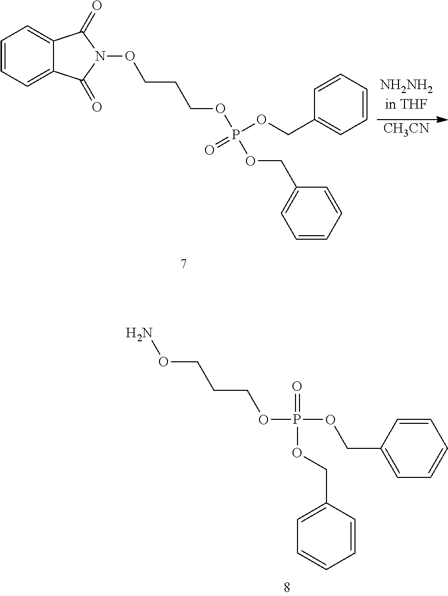

The purified product (7) was dissolved in acetonitrille and an equimolair concentration of hydrazine in tetrahydrofuran was added. The crude 3-(aminooxy)propyl dibenzyl phosphate (8) was formed and solvents were evaporated under a nitrogen stream.

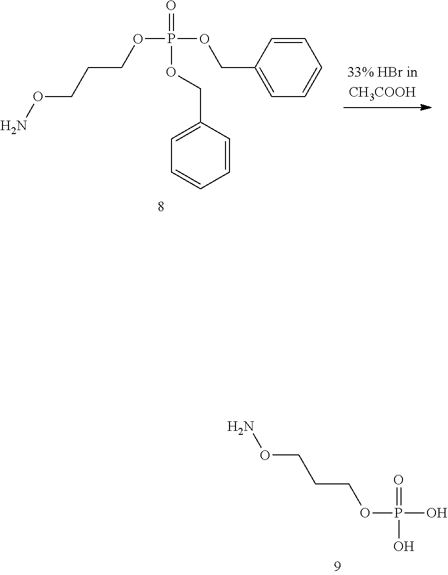 [SMILES: NOCCCOP(=O)(OCc1ccccc1)OCc1ccccc1 ; NOCCCOP(=O)(O)O]

Without purification, the crude 3-(aminooxy)propyl dibenzyl phosphate (8) was fortified with 33% HBr in acetic acid. After 0.5 hour incubation at room temperature, solvents were evaporated under a nitrogen stream. The crude product was dissolved in water and washed 3 times with chloroform. The purified 3-(aminooxy)propyl dihydrogen phosphate (9) was obtained from the water layer, and verified by ¹H-NMR (FIG. 2).

### Example 4

### Synthesis of 4-oxobutyl dihydrogen phosphate

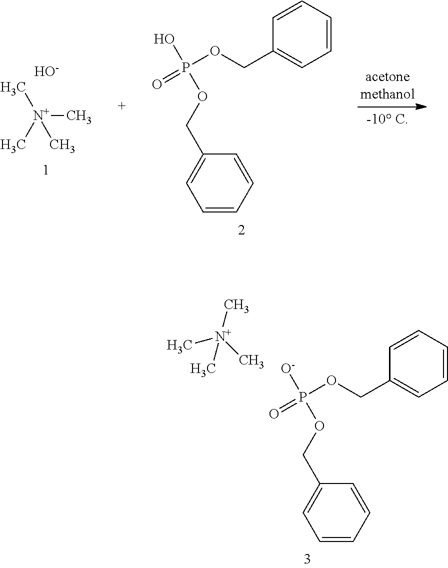

To a freshly prepared mixture of dibenzylphosphate/acetone (2), a mixture of tetramethylammonium hydroxide/methanol/water (1) was added dropwise at −10 C. Solvents were evaporated under a nitrogen stream.

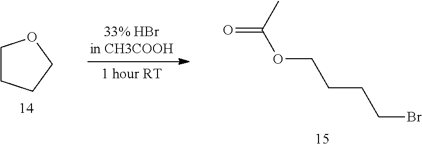

A 33% HBr solution in acetic acid was added to tetrahydrofuran (14). After 1 hour stirring at room temperature, solvents were evaporated under a gentle nitrogen stream. The pure 4-bromobutyl acetate (15) has been obtained.

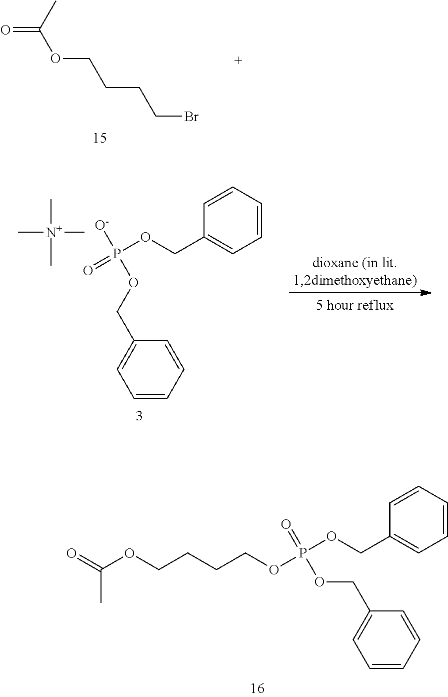

Tetramethylammonium dibenzylphosphate (3) was added to a solution of 4-bromobutyl acetate (15) in dioxane. The mixture was refluxed for 5 hours and dichloromethane was added. The reaction mixture was washed with water and dried with MgSO₄. Volatiles were removed and the resulting crude product dibenzyl 4-acetatebutyl phosphate (16) was purified on silica.

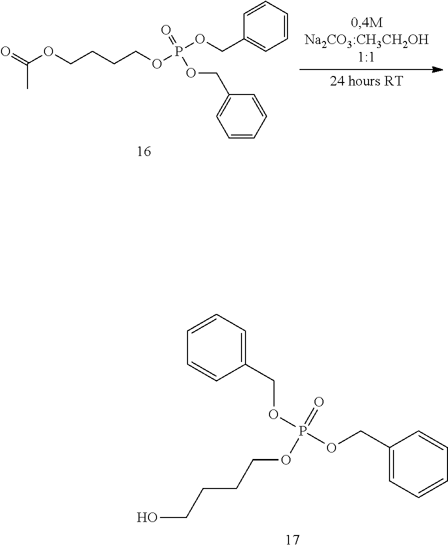 [SMILES: CC(=O)OCCCCOP(=O)(OCc1ccccc1)OCc1ccccc1 ; O=P(OCCCCO)(OCc1ccccc1)OCc1ccccc1]

The pure dibenzyl 4-acetatebutyl phosphate (16) was dissolve in an equimolar concentration of 0.4 M Na₂CO₃ in water and ethanol. After 24 hours stirring at room temperature the mixture was extract with dichloromethane. After drying with MgSO₄, solvents were evaporated under a nitrogen stream. The pure dibenzyl 4-hydroxybutyl phosphate (17) has been obtained.

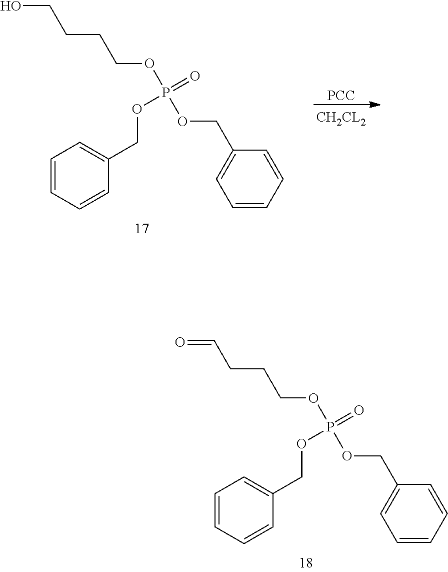 [SMILES: O=P(OCCCCO)(OCc1ccccc1)OCc1ccccc1 ; "PCC" ; O=CCCCOP(=O)(OCc1ccccc1)OCc1ccccc1]

The pure dibenzyl 4-hydroxybutyl phosphate (17) was dissolve in dichloromethane and pyridinium chlorochromate was added. After 1 hour stirring at room temperature the solvents were evaporated under a nitrogen stream and the resulting crude product dibenzyl-4-oxobutyl phosphate (18) was purified on silicagel.

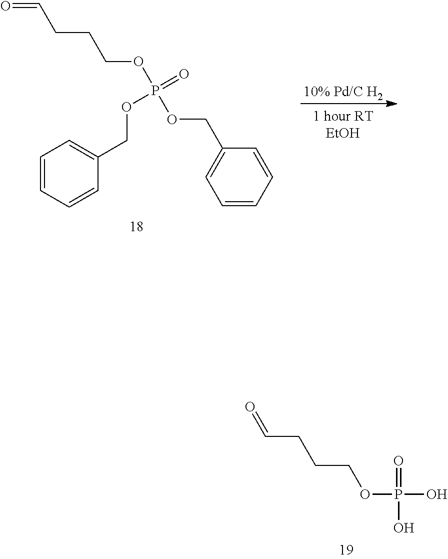 [SMILES: O=CCCCOP(=O)(OCc1ccccc1)OCc1ccccc1 ; "10% Pd/C H2" ; O=CCCCOP(=O)(O)O]

The pure dibenzyl-4-oxobutyl phosphate (18) was dissolve in ethanol and a spatula tip of 10% palladium on carbon was added. The flask was completely filled with hydrogen gas using a dedicated balloon. The solution was stirred at room temperature for 1 hour. The palladium on carbon was filtered off and solvents were evaporated under nitrogen stream. Pure 4-oxobutyl dihydrogen phosphate (19) has been obtained and verified by ¹H-NMR (FIG. 3).

### Example 5

### Synthesis of 4-[(2,5-dioxopyrrolidin-1-yl)oxy]-4-oxobutyl dihdryogen phosphate

The intermediate product dibenzyl 4-hydroxybutyl phosphate of the synthesis route of 4-oxobutyl dihydrogen phosphate (as described above), was used as starting product for the preparation of 4-[(2,5-dioxopyrrolidin-1-yl)oxy]-4-oxobutyl dihydrogen phosphate.

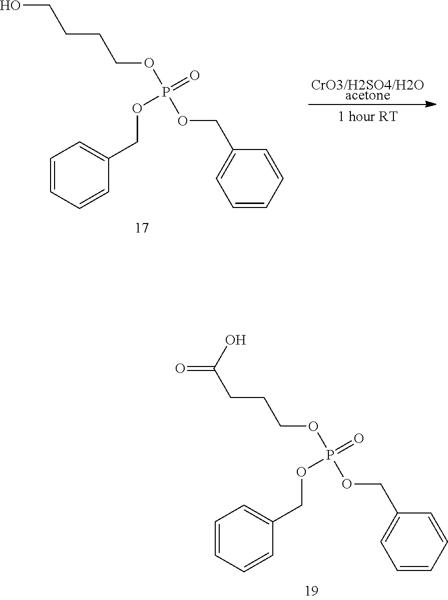 [SMILES: O=P(OCCCCO)(OCc1ccccc1)OCc1ccccc1 ; "CrO3/H2SO4/H2Oacetone" ; O=C(O)CCCOP(=O)(OCc1ccccc1)OCc1ccccc1]

The pure dibenzyl 4-hydroxybutyl phosphate (17) was dissolve in a mixture of chromium (VI) oxide, sulfuric acid, acetone and water. After 1 hour stirring at room temperature 2-propanole was added to inactivate the excess of chromium (VI) oxide. After 1 hour of stirring, dichloromethane and water was added. The 4-{[bis(benzyloxy)phosphoryl]oxy}butanoic acid (19) was obtained from the organic layer with together small amount chromium salts.

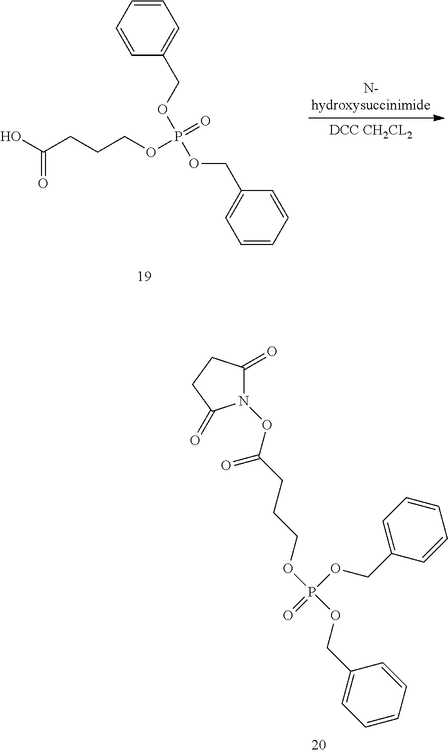 [SMILES: O=C(O)CCCOP(=O)(OCc1ccccc1)OCc1ccccc1 ; O=C(CCCOP(=O)(OCc1ccccc1)OCc1ccccc1)ON1C(=O)CCC1=O]

The crude 4-{[bis(benzyloxy)phosphoryl]oxy}butanoic acid (19) was mixed with N-hydroxysuccinimide, dicyclohexylcarbodiimide in dichloromethane and stirred at room temperature overnight. Solvent were evaporated under nitrogen stream and the resulting crude 4-[(2,5-dioxopyrrolidin-1-yl)oxy]-4-oxobutyl dibenzyl phosphate (20) was purified on silica gel.

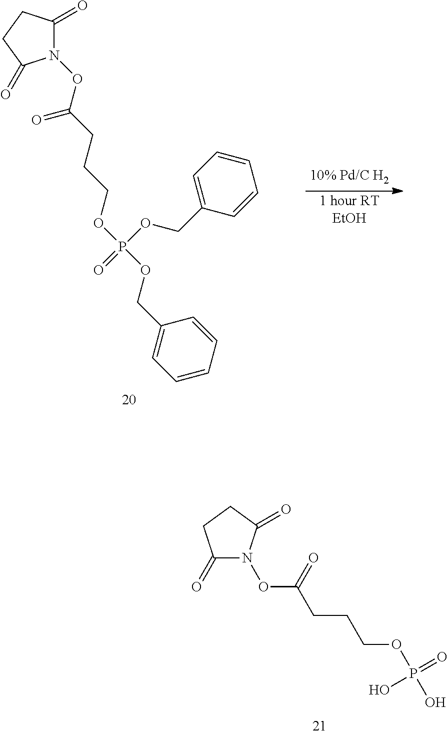 [SMILES: O=C(CCCOP(=O)(OCc1ccccc1)OCc1ccccc1)ON1C(=O)CCC1=O ; O=C(CCCOP(=O)(O)O)ON1C(=O)CCC1=O]

The pure 4-[(2,5-dioxopyrrolidin-1-yl)oxy]-4-oxobutyl dibenzyl phosphate (20) was dissolve in ethanol and a spatula tip of 10% palladium on carbon was added. The flask completely filled with hydrogen gas using a dedicated balloon. The solution was stirred room temperature for 1 hour. The palladium on carbon was filtered off and the solvents were evaporated under nitrogen stream. The pure 4-[(2,5-dioxopyrrolidin-1-yl)oxy]-4-oxobutyl dihydrogen phosphate (21) has been obtained and verified by ¹H-NMR (FIG. 4).

### Example 6

### Selective Isolation of N-Terminal PTAG Modified Model Peptide Using TiO2 Affinity Chromatography

Primary amine coding, using numerous different reagents, is extensively used in comparative proteomics because primary amino groups are easily to deritivatize. Coding can be achieved at α-amino group of the protein, ε-amino groups on lysine or on N-terminal peptides after proteolysis. To demonstrate the concept of this invention, the N-terminal α-amine of standard peptide Oxytocin (CTIGNCPLG) was PTAG modified. Isolation of the modified PTAG-Oxytocin was performed using a TiO₂ online 2D-LC-MS approach (Pinkse, Uitto et al. 2004). Such configuration has been introduced to study protein phosphorylation, which is an important posttranslational modification (PTM) regulatory mechanism where the enzymes kinases (phosphorylation) and phosphatases (dephosphorylation) are involved. However, here it is demonstrated that the concept of TiO2 enrichment is a highly efficient method to isolate a PTAG modified model peptide.

Two sample reaction mixture were prepared, consisting of 10 pmol/μl Oxytocin (Sigma) (CTIGNCPLG), 50 mM K₂HPO₄ pH=7.5 buffer (Sigma), 30 mM NaCNBH₃ (Sigma) and either 4-oxobutyl dihydrogen phosphate or 4-oxo(1,1,2,2-²H₄)butyl dihydrogen phosphate. The sample was incubated for 2 hour at room temperature. Both samples were combined and analyzed using an TiO2 based 2D-LC-MS (Pinkse, Uitto et al. 2004) setup using a QTOF Ultima (Waters) mass spectrometer connected to an Agilent 1100 series. FIG. 5 depicts the experimental conditions.

The results of the selective enrichment of the PTAG model peptide Oxytocin using the online TiO₂/C18 trapping column is shown in FIG. 6. The upper panel shows the ion trace (MH²⁺=580.3 Da and MH²⁺=582.3 Da) of the flowtrough analysis. During that run, the PTAG modified Oxytocin is trapped onto the C18 upstream bed and subsequently eluted using a H₂O/Acetonitrill gradient. Notice the PTAG modified Oxytocin is nicely retained on TiO₂ since its trace is absent in the flowtrough analysis. Subsequently, the PTAG modified Oxytocin is desorbed from the TiO₂ using the alkaline elution buffer (pH of 9), re-concentrated on the downstream C18 and analyzed using a second H₂O/Acetonitrill gradient. From the lower panel it is clear that PTAG modified Oxytocin is successfully desorbed from the TiO₂ and eluted from the C18 column. Furthermore, the ion traces of the light (MH⁺=1159.7 Da) and heavy (MH⁺=1163.7Da) form are depicted in the lower panel of FIG. 2. No or minimal effect of the incorporated isotopes has been observed during the chromatography since both traces co-elute nicely. The mass spectrum of both light (d0) and heavy (d4) N-terminal PTAG modified Oxytocin is depicted in the right panel of FIG. 6.

### Example 7

### Selective Isolation of Cysteine PTAG Modified Peptides from Bovine Serum Albumin BSA Using TiO₂ Affinity Chromatography

A selective modification, isolation and quantification method for cysteine containing peptides is the ICAT and cICAT method, introduced by the Aebersold group (Gygi, et. Al. 1999). In their approach a thiol reactive idoacetamide derivate, having an isotope coded spacer and a biotin affinity tag was developed. Significant reduction in sample complexity was achieved by isolation of the relative rare cysteine containing peptides using the biotin affinity tag. In line with group of Aebersold, the present inventors have developed a selective modification, isolation and quantification method for cysteine containing peptides based on PTAG methodology.

In this example, the utility of the PTAG method to selectively isolate, differentially isotope labeled cysteine containing peptides from Bovine Serum Albumin digest is demonstrated.

Two sample reaction mixture were prepared, consisting of 50 μg Bovine Albumine Serum (BSA), 50 mM K₂HPO₄ pH=7.5 buffer (Sigma). Sulfur bridges were reduced using 10 mM dithiotreitol (Sigma) for 30 min at 37° C. Subsequently, 3-[(bromoacetyl)amino]propyl dihydrogen phosphate or the isotopic analogue 3-[(bromoacetyl)amino](²H₆)propyl dihydrogen phosphate was added to the mixture and the sample was further incubated for 5 hours at 37° C. in addition of 10 mM NaI2. Samples were combined, purified by acetone precipitation and digested by trypsin (1:20 w:w ratio). Analysis was performed by two dimensional TiO2 based LC-MS setup using a QTOF Ultima (Waters) mass spectrometer connected to an Agilent 1100 series. FIG. 5 depicts the experimental conditions. In addition, LC-pseudeMS3 was performed on the sample digest to identify PTAG modified peptides using an Orbitrap XL mass spectrometer.

The results of the selective enrichment of the PTAG model peptide Oxytocin using the online TiO₂/C18 trapping column is shown in FIG. 7. The upper panel depicts base peak ion traces of the flowthrough analysis. During that run, the modified as well as the unmodified peptides are trapped onto the C18 upstream bed and subsequently eluted using a H₂O/Acetonitrill gradient. The unmodified peptides are eluted from the reversed phase column and analyze by mass spectrometry, while PTAG modified cysteine containing peptides are retained onto the TiO₂ bed. Subsequently, the PTAG modified cysteine peptides are desorbed from the TiO₂ using the alkaline elution buffer (pH of 9), re-concentrated on the downstream C18 and analyzed using a second H₂O/Acetonitrill gradient. From the lower panel it is clear that PTAG modified peptides are successfully desorbed from the TiO₂ and eluted from the C18 column. Notice a significant reduction in sample complexity, when comparing flowthrough (unmodified) and PTAG elution analysis. The PTAG modified peptides in the elution analysis could be identified by the d0/d6 isotopic pair as illustrated from the left panel of FIG. 7. In addition, pseudoMS3 analysis in combination with database searching was performed for sequence identification of the PTAG modified peptides. Table 1 provides a list of 16 PTAG modified cysteine containing peptides which were identified by pseudoMS3 fragmentation analysis. Both light (d0) and heavy (d6) forms were identified by database searching.

TABLE 1
Sequence identification of light (d0) and heavy (d6) PTAG labeled
cysteine containing peptides of Albumin (Bovine Serum) after
Titanium Dioxide enrichment and pseudeMS3 data dependent analysis.

| Protein | Peptide sequence identified^{a} | MH+ |
|---|---|---|
| Albumin (Bovine Serum) | C@C@TKPESER | 1442.51 |
| | C\hat{ }C\hat{ }TKPESER | 1454.58 |
| | TC@VADESHAGC@EK | 1739.60 |
| | TC\hat{ }VADESHAGC\hat{ }EK | 1751.68 |
| | SHC@IAEVEK | 1210.52 |
| | SHC\hat{ }IAEVEK | 1216.55 |
| | YIC@DNQDTISSK | 1581.65 |
| | YIC\hat{ }DNQDTISSK | 1587.69 |
| | C@TESLVNR | 1116.47 |
| | C\hat{ }TESLVNR | 1122.51 |
| | YIC@DNQDTISSK | 1581.65 |
| | YIC\hat{ }DNQDTISSK | 1587.69 |
| | LC@VLHEK | 1036.49 |
| | LC\hat{ }VLHEK | 1042.53 |
| | LKEC@C@DKPLLEK | 1808.80 |
| | LKEC\hat{ }C\hat{ }DKPLLEK | 1820.87 |
| | HADIC@TLPDTEK | 1537.66 |
| | HADIC\hat{ }TLPDTEK | 1543.70 |
| | GAC@LLPK | 896.43 |
| | GAC\hat{ }LLPK | 902.47 |
| | VHKEC@CHGDLLECADDRADLAK | 2636.13 |
| | VHKEC\hat{ }CHGDLLECADDRADLAK | 2642.17 |
| | DDPHAC@YSTVFDK | 1692.66 |
| | DDPHAC\hat{ }YSTVFDK | 1698.70 |
| | RPC@FSALTPDETYVPK | 2018.93 |
| | RPC\hat{ }FSALTPDETYVPK | 2024.97 |
| | C@FSALTPDETYVPK | 1765.77 |
| | C\hat{ }FSALTPDETYVPK | 1771.81 |
| | SLHTLFGDELC@K | 1557.70 |
| | SLHTLFGDELC\hat{ }K | 1563.74 |
| | LFTFHADIC@TLPDTEK | 2045.93 |
| | LFTFHADIC\hat{ }TLPDTEK | 2051.97 |
| | MPC@TEDYLSLILNR | 1862.84 |
| | MPC\hat{ }TEDYLSLILNR | 1868.88 |

^{a}Cysteine residues with light (d0) and heave (d6) PTAG label are donated by respectively @ and \hat{ } signs.

### DESCRIPTION OF THE FIGURES

FIG. 1: Structure verification of synthesis product 3-[(bromoacetyl)amino]propyl dihydrogen phosphate by ¹HNMR (D₂O) δ (ppm) 3.92 (q, 2H, POCH₂), 3.90 (s, 2H, CH₂Br), 3.33 (t, 2H, CH₂N), 1.85 (m, 2H, CCH₂C)

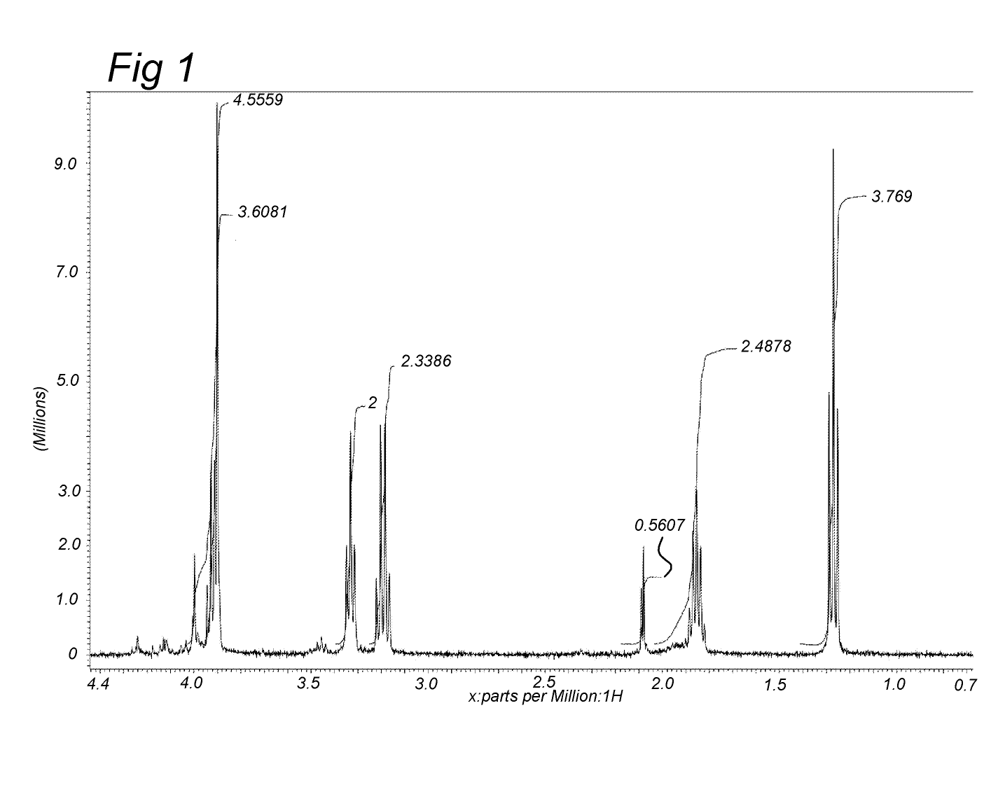

FIG. 2: Structure verification of synthesis product 4-oxobutyl dihydrogen phosphate by ¹HNMR (acetone d6) δ (ppm) 3.98 (q, 2H, POCH₂), 2.58 (t, 2H, CH₂CHO),

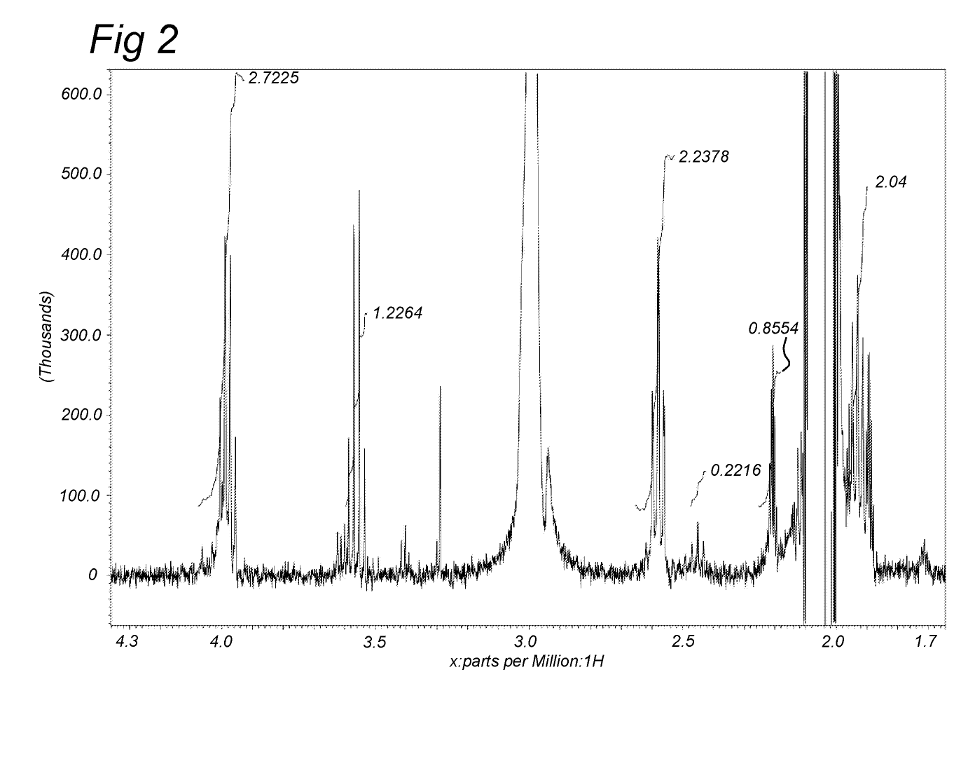

1.93 (m, 2H, CCH₂C) 9.76 (s, 1H, CHO)

FIG. 3: Structure verification of synthesis product 3-(aminooxy)propyl dihydrogen phosphate by ¹HNMR (D₂O) δ (ppm) 4.06 (t, 2H, CH₂ON) 3.90 (q, 2H, POCH₂), 1.89 (m, 2H, CCH₂C).

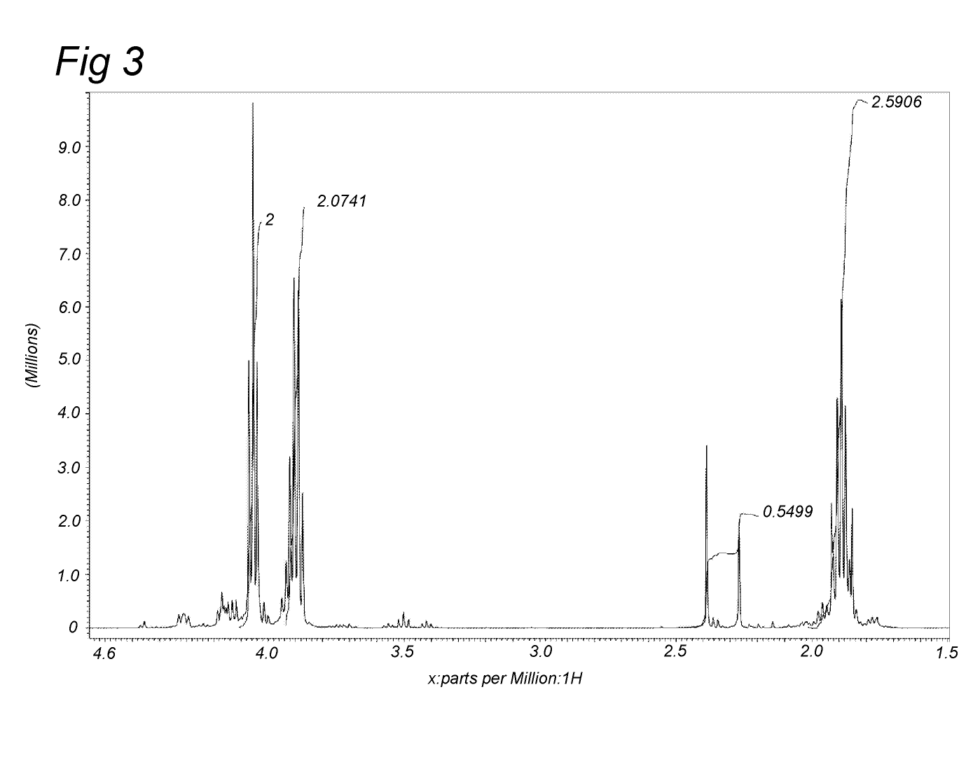

FIG. 4: Structure verification of synthesis product 4-[(2,5-dioxopyrrolidin-1-yl)oxy]-4-oxobutyl dihydrogen phosphate by ¹HNMR (D₂O) δ (ppm) 3.90 (q, 2H, POCH₂), 2.78 (s, 4H, COCH₂CH₂CO), 2.49 (t, 2H, CH₂COO), 1.92 (m, 2H, CCH₂C)

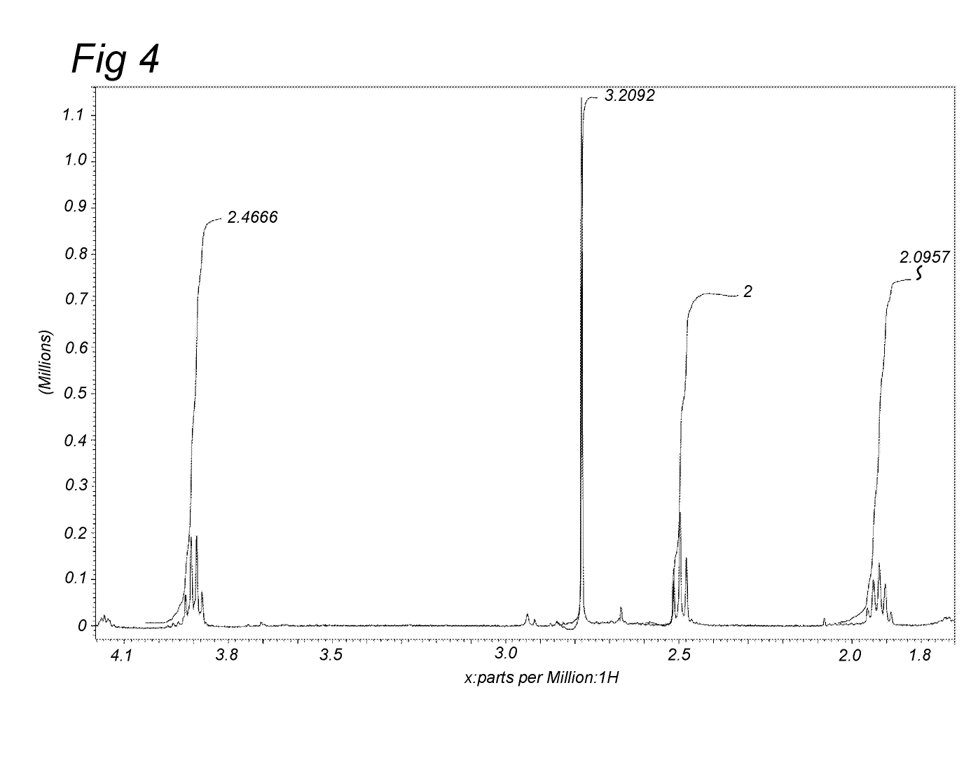

FIG. 5: Schematic representation of the 2D-LC-MS setup. During sample loading, peptides, including PTAG modified peptides are trapped onto the 2D precolumn. In the first H2O/acetonitrile gradient, the non tagged peptides are eluted from the precolumn and analytically separated on the C18 analytical column, while the PTAG modified peptides are retained on the TiO₂. After this first analysis, PTAG modified peptides are desorbed from the TiO₂ precolumn using an alkaline elution buffer with a pH of 9. A second H₂O/acetonitrile gradient is used to analytically analyze the PTAG modified peptides.

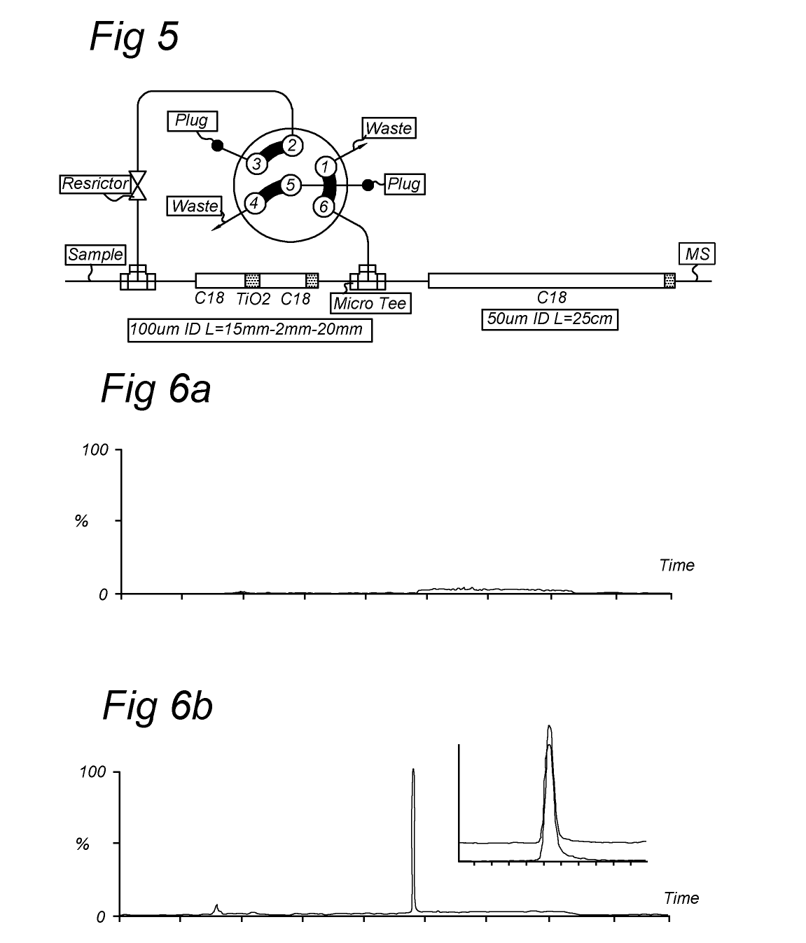

FIG. 6: Specific ion traces of the light (d0) and heavy (d4) N-terminal PTAG modified Oxytocin (MH⁺=1159.7, MH⁺=1163.7 Da). FIG. 6a depicts the flow through analysis of the reaction mixture after loading the sample onto a C18/TiO₂/C18 precolumn. FIG. 6b shows the elution analysis of PTAG modified Oxytocin using the alkaline elution buffer (pH of 9). Moreover, FIG. 6b depicts the co-elution of light (d0) and heavy (d4) N-terminal PTAG modified Oxytocin. The mass spectrum is shown in FIG. 6c.

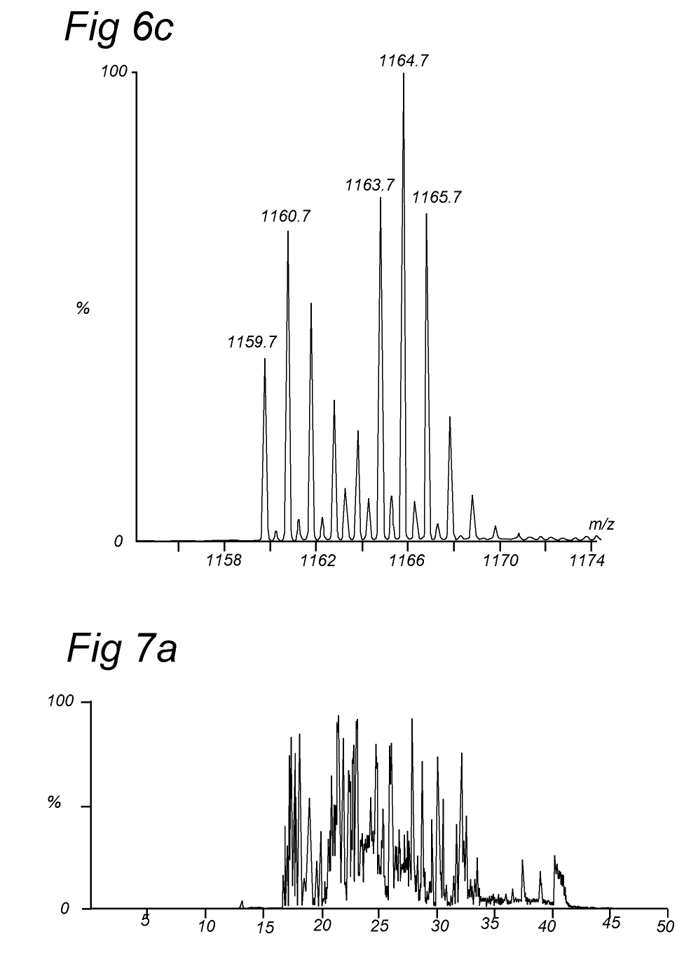

FIG. 7: Base peak ion traces of the light (d0) and heavy (d6) PTAG-labeled cysteine containing peptides of Albumin (Bovine Serum) after trypsination. FIG. 7a depicts the flowthrough analysis of the unmodified peptides after loading the sample onto a C18/TiO₂/C18 precolumn. FIG. 7b shows the elution analysis of PTAG modified cysteine containing peptides using the alkaline elution buffer (pH of 9). FIG. 7c illustrates the d0/d6 doublet of a PTAG labeled cysteine containing peptide originating as a MH2+ ion in the mass spectrum.

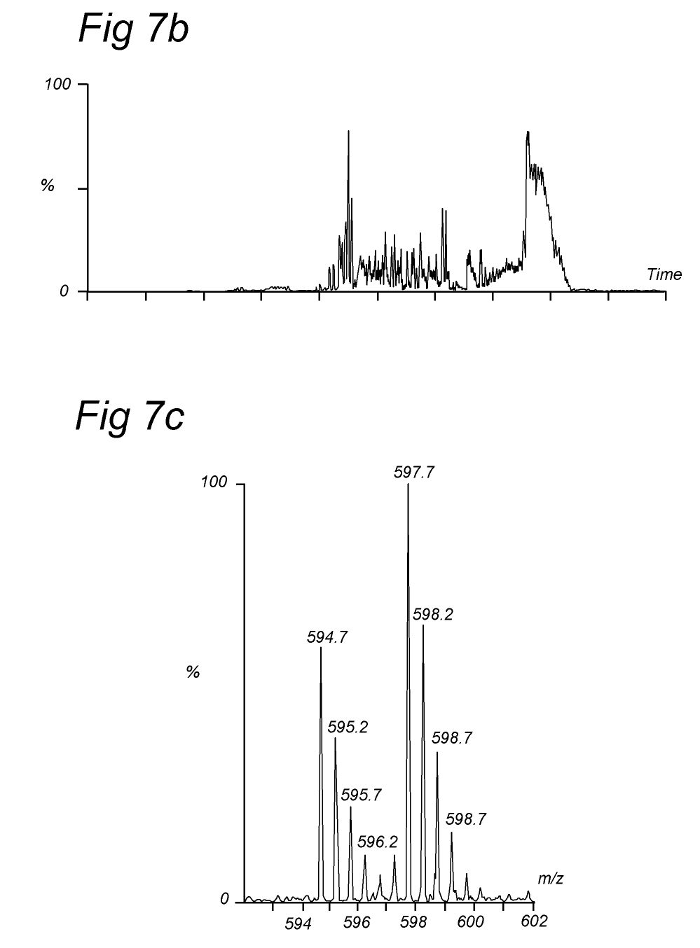

1. An organophosphate compound having the general structure:
X—SPACER-O—PO₃H₂
wherein:
X is a reactive group which is reactive towards a chemical moiety on a protein, a peptide, a nucleic acid molecule, a lipid, a carbohydrate and/or a steroid, and
‘Spacer’ represents a chain consisting of 1-5 building blocks selected from:
—(CH₂)ₙ—, wherein n is an integer of 1-5 and wherein one or more hydrogen atoms may have been replaced with a substituent independently selected from, —R, —OR, ═O, ═NR, ═N₂, ═N—O—R, —NRR′, —SR, -halogen, —OC(O)R, —C(O)R, —CO₂R, —CONRR′, —OC(O)NRR′, —NRC(O)R′, —NR—C(O)NR″, R′″, —NC(O)₂R, —NR—C(NRR″R′″)═NR″″, —NR—C(NRR″)═NR′″, —SOR, —S(O)₂R, —S(O)₂NRR″, —NRSO₂R′, —CN, —O—PO₃H₂ and —NO₂, wherein R, R′, R″ and R′″ independently represent hydrogen or a lower branched or linear alkyl, alkenyl, alkynyl, alkoxy, thioalkoxy, heteroalkyl, cycloalkyl, cycloalkenyl, aryl, cycloalkylalkyl, cycloalkenylalkyl, or arylalkyl moiety; or with a fluorophore or a chromophore;
-Phe-, wherein one or more hydrogen atoms may have been replaced with a substituent selected from —R, —OR, —NRR′, —SR, -halogen, —OC(O)R, —C(O)R, —CO₂R, —CONRR′, —OC(O)NRR′, —NRC(O)R′, —NR—C(O)NR″, R′″, —NC(O)₂R, —NR—C(NRR″R′″)═NR″″, —NR—C(NRR″)═NR′″, —SOR, —S(O)₂R, —S(O)₂NRR″, —NRSO₂R′, —CN, —O—PO₃H₂ and —NO₂, wherein R, R′, R″ and R′″ independently represent hydrogen or a lower branched or linear alkyl, alkenyl, alkynyl, alkoxy, thioalkoxy, heteroalkyl, cycloalkyl, cycloalkenyl, aryl, cycloalkylalkyl, cycloalkenylalkyl, or arylalkyl moiety;
—CH═CH—, wherein one or two hydrogen atoms may have been replaced with a substituent selected from —R, —OR, —NRR′, —SR, -halogen, —OC(O)R, —C(O)R, —CO₂R, —CONRR′, —OC(O)NRR′, —NRC(O)R′, —NR—C(O)NR″, R′″, —NC(O)₂R, —NR—C(NRR″R′″)═NR″″, —NR—C(NRR″)═NR′″, —SOR, —S(O)₂R, —S(O)₂NRR″, —NRSO₂R′, —CN, —O—PO₃H₂ and —NO₂, wherein R, R′, R″ and R′″ independently represent hydrogen or a lower branched or linear alkyl, alkenyl, alkynyl, alkoxy, thioalkoxy, heteroalkyl, cycloalkyl, cycloalkenyl, aryl, cycloalkylalkyl, cycloalkenylalkyl, or arylalkyl moiety;
—C≡C—;
—O—;
—S—; and
—NR—, wherein R indepenently represents hydrogen or a lower branched or linear alkyl, alkenyl, alkynyl, alkoxy, thioalkoxy, heteroalkyl, cycloalkyl, cycloalkenyl, aryl, cycloalkylalkyl, cycloalkenylalkyl, or arylalkyl moiety;
and, optionally, a building block selected from:
—C(O)—O—SiRR′— and —O—SiRR′, wherein R, and R′ independently represent hydrogen or a lower branched or linear alkyl, alkenyl, alkynyl, alkoxy, thioalkoxy, heteroalkyl, cycloalkyl, cycloalkenyl, aryl, cycloalkylalkyl, cycloalkenylalkyl, or arylalkyl moiety;
—S—S—;
—O—PO₂—O—; and
—S(═O)(═O)—;
or ‘Spacer’ represents a moiety comprising:
one or multiple nucleic acid bases;
a protease or enetrokinase cleavage sequence;
a lactamase sensitive P-lactam analogue;
a thrombin cleavage sequence;
a glycosidase-cleavable sugar; or
a nuclease cleaving sequence;
wherein each Spacer moiety may comprise an optically detectable moiety as a substituent;
wherein at least one atom in at least one of the Spacer moiety, —X and the phosphate moiety has the form of a stable isotope of the corresponding element;
and salts and esters thereof.

2. The organophosphate compound according to claim 1, wherein the spacer comprises at least one heteroatom and has a main chain with a length of 20 atoms or less.

3. The organophosphate compound according to claim 1, wherein the spacer does not comprise heteromatoms and has a main chain with a length of 10 atoms or less.

4. The organophosphate compound according to claim 1, wherein the spacer comprises a peptide chain of at least 3 consecutive amino acids.

5. The organophosphate compound according to claim 1, wherein said stable isotope is selected from the group consisting of ²H, ¹³C, ¹⁵N, ¹⁸O, ³²P, ³³P and ³⁴S.

6. The organophosphate compound according to claim 1, wherein at least one atom in at least one of the X and Spacer moieties is a stable isotope.

7. The organophosphate compound according to claim 1, wherein X is
a thiol reactive group selected from halo acetylamino, haloacetyls, α-haloacyls, maleimido, pyridyl disulfides, pyridylthio propionate, chloroacetylamino, phenylmercury, phenyllead, phenylcadmium, thiosulfates, thiomethylsulfones, arylating a agents, epoxides, nitrifies, aziridines, acryloyl, alkyl and aryl halides, thiol-disulfide exchange reagents;
an amine reactive group selected from succinimidyl esters, sulfosuccinimidyl esters, tetrafluorophenyl esters, and sulfodichlorophenol esters, isothiocyanates, isocyanates, sulfonyl halides, dichlorotriazines, arylating agents, aryl halides, acyl azides, carboxylic acids, carbonates, anhydrides, epoxides, ketones, alehydes, imidoesters and carbodiimides;
a carboxylic acid reactive groups selected from hydrazines, hydroxylamines, primary aliphatic amines, primary aromatic amines, semicarbazides, diazoalkanes, diazoacetyls, carbonyldiimidazole, carbohydrazide, carbodiimides and 2,3,5,6-tetrafluorphenyl trifluoroacetate;
a hydroxyl reactive group selected from epoxide, carbonyldiamidazole, N,N-disuccinimidyl carbonate, N-hydroxysuccinimidyl chlorofromate, dichlorotriazine, boronic acids, isocyanates, acyl nitriles, alkyl halogens and amine- or hydrazine reactive groups; or
an aldehyde or ketone reactive group selected from hydrazines, semicarbazides, carbohydrazide, hydroxylamines, primary aliphatic amines and primary aromatic amines.

8. The organophosphate compound according to claim 1, wherein said spacer comprises a cleavable linker, selected from
photo cleavable groups,
thermally cleavable groups, preferably one or more nucleic acid bases;
chemically cleavable linkers,
enzyme-cleavable linkers,
linkers that are nuclease-cleavable; and
linkers that are glycosidase-cleavable;

9. The organophosphate compound according to claim 1, wherein said spacer is a linear C₃-C₅ alkyl.

10. The organophosphate compound according to claim 1 selected from the group of:
3-[(bromoacetyl)amino](²H₆)propyl dihydrogen phosphate;
4-[(2,5-dioxopyrrolidin-1-yl)oxy]-4-oxo(²H₆)butyl dihydrogen phosphate;
4-oxo(1,1,2,2-²H₄)butyl dihydrogen phosphate;
3-(aminooxy)(²H₆)propyl dihydrogen phosphate;
3-amino(²H₆)propyl dihydrogen phosphate;
and salts and esters thereof.

11. A method of enriching for one or more target molecules in a sample, said method comprising the steps of:
a) providing said sample comprising said one or more target molecules;
b) combining the sample with a composition comprising an organophosphate compound as defined according to claim 1;
c) subjecting the sample to conditions that allow for selective binding of the organophosphate compound to the one or more target molecules;
d) separating organophosphate compound bounded substances from unbound substances, using a separation technique based on selective phosphate binding, calcium phosphate precipitation, phosphoramidate chemistry or isoelectric focussing, yielding a fraction enriched in the one or more target molecules.

12. The method according to claim 11, further comprising the step of subjecting the sample to conditions resulting in cleavage of the phosphate moiety from the target molecule or to conditions resulting in hydrolysis of the phosphate moiety following step d).

13. The method according to claim 11, further comprising the step of-subjecting the enriched sample to mass spectrometric analysis.

14. A method of labeling a target molecule, comprising reacting an organophosphate compound according to claim 1, wherein target molecule is selected from the group consisting of nucleic acids, carbohydrates, proteins, peptides, lipids and steroids, contained in a sample.

15. A peptide or protein comprising, within its amino acid sequence and/or at its N-terminal and/or C-terminal positions, one or more building blocks represented by the formula: 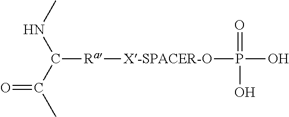
Wherein:
—R^{a′} represents the residue of an amino acid side chain,
—X′— represents the residue of a reactive group X; and
X and -Spacer- have the same meaning as defined in claim 1.

16. A kit comprising a first container and a second container, said first container holding a quantity of a first organosphosphate compound as defined in claim 1; and said second container holding a quantity of a organophosphate compound that is chemically identical but isotopically different from said first organophosphate compound.

17. The organophosphate compound according to claim 1, wherein X is a thiol reactive group, an amino reactive group, a carboxylic acid reactive group, a hydroxyl reactive group, or an aldehyde or ketone reactive group.

18. The organophosphate compound according to claim 8, wherein the photo cleavable groups are selected from the group consisting of O-nitrobenzyl, desyl, trans-o-cinnamoyl, and m-nitrophenyl, benzylsulfonyl groups

19. The organophosphate compound according to claim 8, wherein the thermally cleavable groups comprise one or more nucleic acid bases.

20. The organophosphate compound according to claim 8, wherein the chemically cleavable linkers are selected from the group consisting of diols, diazo, esters, sulfone, —S—S—, diarylmethyl or trimethylarylmethyl groups, silyl esters, carbamates, oxyesters, thioesters, thionoesters, and fluorinated amines.
